(19) United States
(12) Patent Application Publication
(10) Pub. No.: US 2025/0318518 A1
(43) Pub. Date: Oct. 16, 2025

(54) CELL-SEEDED SUBSTRATES AND RELATED MATERIALS AND METHODS

(71) Applicant: Regenerative Patch Technologies LLC, Portola Valley, CA (US)

(72) Inventors: Nicholas Scianmarello, Monrovia, CA (US); Adam Rifat Akkad, Santa Barbara, CA (US); Britney Ocean Pennington, Goleta, CA (US); Mohamed Faynus, Brampton (CA); Jason Shih, Yorba Linda, CA (US)

(21) Appl. No.: 19/176,845

(22) Filed: Apr. 11, 2025

Related U.S. Application Data
(60) Provisional application No. 63/633,509, filed on Apr. 12, 2024.

(51) Int. Cl. A01N 1/125; B26B 5/00; B26B 25/00

(52) U.S. Cl. CPC A01N 1/125; B26B 5/006; B26B 25/005

(57) ABSTRACT

The present disclosure provides devices, instruments, and kits for handling certain implantable substrates, for example, cell-seeded membranes. The devices, instruments, and kits may be used for washing, cutting, and preparing the substrates for implantation into a patient. The devices, instruments, and kits may be particularly useful for handling implantable substrates that were cryopreserved.

### CROSS-REFERENCE TO RELATED APPLICATIONS

This application claims priority to U.S. Provisional Patent Application No. 63/633,509, filed Apr. 12, 2024, which is incorporated herein by reference in its entirety.

### BACKGROUND

The scope of human disease that involves loss of, or damage to, cells is vast and includes, but is not limited to, ocular disease, neurodegenerative disease, endocrine disease, cardiovascular disease, and cancers. Cellular therapy involves the use of cells to treat diseased or damaged tissues. It is rapidly coming to the forefront of technologies that are poised to treat many diseases, in particular those that affect individuals who are non-responsive to traditional pharmacologic therapies. Many of these diseases would benefit from long-term, concentrated target-area treatment, which would reduce systemic side effects. Furthermore, certain drugs such as protein therapeutics are expensive, costing thousands of dollars per vial and require repetitive treatments with no end. In fact, many diseases that are candidates for application of cellular therapy are not fatal, but involve loss of normal physiological function. For example, ocular diseases often involve functional degeneration of various ocular tissues and affect the vision, and thus the quality of life, of numerous individuals.

The mammalian eye is a specialized sensory organ capable of converting incoming photons focused by anterior optics (cornea and lens) into a neurochemical signal. This process of phototransduction facilitates sight by sending action potentials to higher cortical centers via the optic nerve. The retina of the eye comprises photoreceptors that are sensitive to various levels of light and interneurons that relay signals from the photoreceptors to the retinal ganglion cells. These photoreceptors are the most metabolically active cells in the eye (if not in the body), and are supported metabolically and functionally by retinal pigment epithelial (RPE) cells. These RPE cells are organized in a monolayer in the eye and are critical to vision.

Numerous pathologies can compromise or entirely eliminate an individual's ability to perceive visual images, including trauma to the eye, infection, degeneration, vascular irregularities, and inflammatory problems. The central portion of the retina, known as the macula, is responsible for central vision, fine visualization, and color differentiation. The function of the macula may be adversely affected by age-related macular degeneration (wet or dry), diabetic macular edema, idiopathic choroidal neovascularization, high myopia macular degeneration, or advanced retinitis pigmentosa, among other pathologies.

Age-related macular degeneration typically causes a loss of vision in the center of the visual field (the macula) because of damage to the retina. It is a major cause of visual impairment in adults over 50 years of age. Macular degeneration occurs in “wet” and “dry” forms. In the dry form, cellular debris (drusen) accumulates between the RPE cell layer and the choroid, adversely affecting the RPE cells and leading to their dysfunction, degeneration and, ultimately, death. The photoreceptor cells of the retina that depend on viable RPE cells to perform crucial support functions become dysfunctional and die as a secondary effect of the RPE cell pathology. In the more severe wet form of macular degeneration, newly formed blood vessels from the choroid infiltrate the space behind the macula; the newly formed blood vessels are fragile and often leak blood, causing the death of photoreceptors and their supporting cells.

While diseases that cause damage to specific cells or tissues are clear candidates for cellular therapy, there remains a need in the art for improved methods of cellular therapy—in particular, methods, substrates, and tools to improve the efficacy of cellular therapy, as well as methods and compositions allowing for long-term storage of functional and viable cells to be used in such therapies.

### SUMMARY

In various embodiments, the present disclosure relates generally to methods and compositions for the growth of one or more cell layers on one or more substrates, subsequent cryopreservation, thawing, and preparation for implantation. Particular methods and compositions relate to cells seeded and/or grown on a polymeric substrate. In certain applications, the cells are retinal pigment epithelial (RPE) cells, photoreceptor cells, bipolar cells, amacrine cells, ganglion cells, glial cells, fibroblastic cells, stem cells, pre-differentiated cells, differentiations of such cells, or a combination of one or more of the foregoing cell categories, regardless of the differentiation, derivation or culture history of the cells.

Methods include preparation steps specifically tailored for manufacturing using a substrate including custom plating, cutting, growing, and cryopreserving tooling as well as methods and procedures used to reproducibly produce cellular therapy products.

To address the need for improved long-term storage of cell-containing compositions for use in cell therapy, there is provided, in some embodiments, a method of cryopreserving cells on a substrate comprising one or more layers of differing cell types. In various embodiments, the method involves exposing a substrate, which has been seeded with a composition of cells, to a temperature ramp-down phase having a desired temperature reduction rate; transferring the cell-seeded substrate to a desired intermediate temperature range for a first period of time; and maintaining the cell-seeded substrate at a desired storage temperature range for a second period of time, resulting in cryopreserved cells on a substrate that are suitable for long term storage and later use in cell therapy after thawing. The first and second periods of time may be the same, substantially similar, or different.

The present disclosure provides, in various embodiments, cryopreservation methods and variations of procedural steps to accommodate a variety of cell characteristics. Additionally, specific cell characteristics and substrate characteristics may be selected to improve the cell viability and implant success of the cell-seeded substrate for efficacious treatment. Embodiments of the present disclosure provide new methods and apparatus that offer advantages including beneficial outcomes for cryopreservation, subsequent thawing, and various preparation steps of cell-seeded substrates—e.g., higher cell survival, minimal damage to substrates, and a simplified process for generating cell-seeded substrates suitable for cell growth and direct implantation.

In some embodiments, the present disclosure provides one or more pedestals that may be configured to receive and temporarily hold an implantable substrate (e.g., cell-seeded membrane) during steps to prepare the implantable substrate for implantation. These steps may include, for example, washing and/or cutting of the implant. In some embodiments, the pedestal includes a floor and a ramp nook having one or more sidewalls protruding from the floor. The floor, in some embodiments, may provide a horizontal surface on which the implant and/or liquid may be positioned. In some embodiments, the one or more sidewalls of the ramp nook may protrude vertically from the planar floor and at least partially define a cavity within the ramp nook. The cavity may be particularly sized and shape to receive a portion of the implantable substrate. In some embodiments, the cavity of the ramp nook may be sized and shaped to receive only a first portion of the implantable substrate while a second portion of the implantable substrate extends outside of the cavity. The second portion of the implantable substrate may be positioned in a cutting region of the floor outside of the ramp nook, for example, such that the second portion is accessible for cutting. In some embodiments, the cavity includes a sloped bottom surface that is angled obliquely relative to a plane of the floor. In some embodiments, the sloped bottom surface slopes to a point below a plane of the floor to define a well in the floor.

In further embodiments, the pedestal may include one or more dividing walls that also protrude from the floor. In some such embodiments, a dividing wall may at least partially divide the floor into two or more regions, for example, a cutting region on a first side of the dividing wall and a washing region on a second side of the dividing wall. In some embodiments, the sidewalls of the ramp nook may be extend directly from one side of the dividing wall. In some embodiments, the dividing wall may also partially define the cavity of the ramp nook. In some embodiments, the sloped bottom surface of the cavity of the ramp nook may slope upwards as the bottom surface extends away from the dividing wall. In yet further embodiments, the pedestal can include a perimeter containment wall that surrounds a portion or all of the floor. In some embodiments, the pedestal is configured to contain a liquid (e.g., rising/washing solution) in an area bounded by the perimeter containment wall. The perimeter containment wall height may be sufficient to contain a volume of liquid to fully submerge the implantable substrate according to some embodiments. The perimeter containment wall may include indicia or markings to identify the volume of liquid contained in the pedestal. The pedestal may be open at top to allow the implant and/or liquid to be placed on the pedestal from above during use. In some embodiments, the floor may include one or more ports that are configured to introduce and/or remove liquid from below through the floor of the pedestal. The ports may be opened during liquid introduction/removal, and closed to retain liquid in the pedestal (e.g., during implant washing) according to some such embodiments. One or more components of the pedestal (e.g., floor, ramp nook, and/or walls) may be integrally formed with each other such that they are of a seamless, unitary construction. The one or more components may be formed, for example, from a common material (e.g., a plastic material).

In some embodiments, the present disclosure provides a method for preparing an implantable substrate for implantation. The method may include providing a pedestal having a floor with a cutting region and a ramp nook having one or more sidewalls protruding from the floor, the one or more sidewalls at least defining a cavity, placing a first portion of the implantable substrate within the cavity while a second portion of the implantable substrate extends outside of the cavity into the cutting region, and cutting the second portion of the implantable substrate from the first portion of the implantable substrate. In some embodiments, the second portion of the implantable substrate is cut while the first portion of the implantable substrate is within the cavity of the ramp nook. In some examples, the implantable substrate is a cell-seeded membrane. In further embodiments, where the implantable substrate was cryopreserved, the implantable substrate is thawed, submerged in a liquid contained in the pedestal (e.g., on the floor or in the cavity), and rinsed of cryoprotectant prior to cutting the second portion of the implantable substrate.

In still further embodiments, the present disclosure provides one or more tools (e.g., forceps, cutting tools) for manipulating an implantable substrate and kits containing such tools. The kits may further include one or more pedestals as described herein.

The term “substantially” or “approximately” means±10% (e.g., by weight or by volume), and in some embodiments, ±5%. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The term “consists essentially of” means excluding other materials that contribute to function, unless otherwise defined herein. Nonetheless, such other materials may be present, collectively or individually, in trace amounts. The headings provided herein are for convenience only and are not intended to limit or interpret the scope or meaning of the claimed technology.

### BRIEF DESCRIPTION OF THE DRAWINGS

The foregoing will be more readily understood from the following detailed description, in particular, when taken in conjunction with the drawings, in which:

FIG. 1A illustrates progressively enlarged view of a substrate (e.g., membrane) suitable for use in connection with various embodiments of the present disclosure. The substrate may be, for example, seeded with cells to provide a cell-seeded substrate for implantation to treat certain medical conditions as described herein.

FIG. 1B shows the various parts of a substrate for cell-seeding.

FIG. 2A is a partially transparent perspective view of a cell-seeded substrate as shown in FIG. 1 disposed within in a cryovial with no assisted holder.

FIG. 2B illustrates a cell-seeded substrate as shown in FIG. 1 retained by a substrate holder sliding into a standard cryovial.

FIG. 2C shows the substrate holder with retained cell-seeded substrate fully received within the cryovial.

FIGS. 3A-3B are perspective views of a cryopreservation tray according to certain embodiments that is capable of holding multiple cell-seeded substrates.

FIG. 4 shows a pedestal according to some embodiments that is sized and configured to receive a cell-seeded substrate and provide a platform for rising/washing the cell-seeded substrate and/or to allow for clean cutting of the tail portion from the cell-seeded substrate prior to implantation.

FIG. 5A shows a side cross-sectional view of a pedestal according to further embodiments that includes one or more inlet and outlet ports.

FIG. 5B shows an embodiment of a sieve with a repeated pattern of holes that may be included in the substrate holder of FIG. 5A.

FIGS. 6A-6C show a pedestal according to yet another embodiment. The ramp nook according to this embodiment may be specifically shaped to the implant (e.g., the cell-seeded substrate) and may be a proportional sized larger (e.g. 105%-150%, etc.) to allow for easy placement of the implant into the ramp nook.

FIG. 7 shows an additional embodiment of an implant cutting region for a pedestal.

FIG. 8 shows an instrument (e.g., trephine blade) that may be used for surgical removal of certain tissues in a curved fashion according to some embodiments, for example, corneal tissue of the eye. The instrument may include a handle and a circular blade.

FIGS. 9A-9D shows a flat bottom wall forceps having a flat surface that serves the purposes of holding the implant against the pedestal and providing a wall to guide the cutting blade.

FIGS. 10A-10D depict an embodiment in which a sliding cutting blade is integrated in the design of the wall forceps.

FIGS. 11A-11D depict an embodiment in which a cutting blade is integrated in the design of the wall forceps.

### DETAILED DESCRIPTION

The present disclosure relates, generally, to cell-seeded substrates, cell evaluation processes, and methods to increase viability of the cell-seeded membranes through cryopreservation and implantation preparation processes. Cell-seeded membranes, unlike conventional single cells or clusters of cells, require special handling to promote viability through the manufacturing process as well as following cryopreservation and thaw prior to implantation. By specifically making the adaptations described herein, it is possible to increase the manufacturing yield of cell-based therapies as well as survivability of cells and their health (e.g., metabolic activity, longevity).

### Manufacture of Substrates/Membranes

In various embodiments, the substrate comprises, consists essentially of, or consists of a biocompatible polymers such as natural substrates (collagen, bironectin, laminin, various combinations of extracellular matrix components), synthetic substrates (hydrogels, PLGA, silicone, parylene, and other polymers). In some embodiments, the substrate comprises one or more polymers in combination with other materials, the other materials being either biodegradable or non-biodegradable. In an non-exhaustive list, natural substrate materials may consist of one or more of: gelatin, retinal pigmented epithelial extracellular matrix (RPE ECM), Descemet's membrane (porcine, bovine), lens capsule (human, porcine), fibrinogen (crosslinked fibrogen particles), amniotic membrane, Bruch's membrane (human), inner limiting membrane (human), collagen, and other membranes found throughout various tissues in the human body. Synthetic substrate materials may consist of one or more of: Microphotodiode array materials (Tin, SiO2, Si3N4, Ir, Platinum, MPDA-Pt), PMMA PMMA=polymethylmethacrylate, PLGA/P8HVB P8HVB, Thermo-responsive polymer=4-(N-cinnamoylcarbamide) methylstyrene and N-iso, Cryo-precipitate=UV and riboflavin-5 phosphate X linked plasmacryoprecipitate, PDMS (polydimethylsiloxane), Poly-urethanes (Pellethane Tecoflex, Zytor), Agarose and GRGDS GRDS=glycine-arginine-glycine-aspartic acid-serine, ELR and RGD ELR=elastin-like recombinamer RGD=arginine-glycine-aspartic acid, HAMC=hyaluronan and methyl cellulose, IPM=interphotoreceptor matrix (porcine), bFGF=basic fibroblast growth factor, PEG=poly(ethylene glycol), NIPAAm=N-isopropylacrylamide, NAS=N-hydroxysuccinimide, HA=hyaluronic acid, RGD=arginine-glycine-aspartic acid, PCL=PCL=poly(ε-caprolactone) (biodegradable), PCL with Laminin PCL=poly(ε-caprolactone), PCL/PCL PCL=poly(ε-caprolactone), PGS PGS=poly(glycerol-sebacate) biodegradable at intermediate thickness (45 um), PGS and PCL and Laminin PNIPAAm-grafted collagen PGS=poly(glycerol-sebacate), PCL=poly(ε-caprolactone), NIPAAm=N-isopropylacrylamide, PGS PGS=poly(glycerol-sebacate), PGS, RGD, PCL and Laminin PGS=poly(glycerol-sebacate) RGD=arginine-glycine-aspartic acid PCL=poly(ε-caprolactone), PLGA PLGA=poly(lactic-co-glycolic acid), PLGA and MMP2 PLGA=poly(lactic-co-glycolic acid) MMP2=Matrix metalloproteinase 2, PLGA PLLA PLGA=poly(lactic-co-glycolic acid), PLGA-MMP2 PLGA=poly(lactic-co-glycolic acid), PLGA-PHBV8, laminin and poly-L-lysine PLGA=poly(lactic-co-glycolic acid) PHBV8=poly(hydroxybutyrate-co-hydroxyvaleric acid), PLL HA HA=hyaluronic acid, PLL/CSA PLL=poly-L-lysine CSA=chondroitin sulfate, PLL/PSS PLL=poly-L-lysine PSS=*not listed in literature, PLLA and PLGA PLLA=poly(L-lactic acid) PLGA=poly(lactic-co-glycolic acid), PLLA/PLGA PLLA=poly(L-lactic acid) PLGA=poly(lactic-co-glycolic acid), PMMA and Laminin PMMA=polymethylmethacrylate, PS and ECL PS=Polystyrene ECL=enactin collagen and laminin and combinations thereof.

The substrate may be treated such that it has one or more characteristics that enhance viability of the seeded cells. For example, the substrate may further comprise a coating to enhance adhesion of the cells to the substrate. In some embodiments, the coating comprises one or more of Matrigel, vitronectin, and retronectin. Other coatings or surface modifications may be used to achieve improved cell adhesion to the substrate and/or to improve the durability and/or viability of the cells and the substrate during and after the cryopreservation process. For example, in various embodiments, the coating enhances the viability of the cells during cell culture, cryopreservation, after cryopreservation, or both.

In some embodiments, the characteristics of the substrate comprise one or more of the coefficient of thermal expansion of the substrate, a substrate elasticity parameter, or a substrate thickness. The substrate may be selectively permeable and the characteristic may be or comprise substrate thickness; the thickness may be selected to allow nutrients to pass through the substrate. The substrate may have no through holes and rely solely on thickness for permeability. Thus, upon thawing following implantation at a target site, the substrate permits adequate passage of nutrients to the cells and/or adequate passage of cellular waste material away from the substrate. In some embodiments, the thickness is selected to yield a thermal coefficient of expansion of the substrate such that it has minimal or reduced adverse clinical impact on the seeded cells. In some embodiments, the material and thickness are selected to exhibit thermal-energy release characteristics that do not interfere with the release of latent heat by the seeded cells. In some embodiments, the material configuration is selected to have increased sheer force resistance; for example, the configuration may be a hexagonal, honeycomb pattern geometry, reinforced in one or more key areas such as the perimeter, structural columns, or areas connecting multiple layers.

In some embodiments where two or more layers of differing cells are placed on the membrane, a second substrate layer may be placed in between such cell layers to promote segregated proliferation within separate layers. For example, when the cell-seeded substrate has the specific order of (i) a first substrate layer, (ii) a first layer of cells comprising, consisting of or consisting essentially of RPE cells with a basal side interfacing with the substrate, and (iii) a second layer of cells comprising, consisting of, or consisting essentially of photoreceptor cells interfacing with the apical surface of the first layer of cells, a second substrate layer may be disposed between the first and second layers of cells. In one embodiment, the two cell layers are grown on the first substrate layer simultaneously, with the first cell layer of RPE cells being seeded onto the substrate first and the second cell layer of photoreceptors being seeded onto the substrate at a subsequent time. Such time may be 1-10 days later, thereby allowing RPE cells to primarily adhere to the substrate first, mature, and polarize to have apical and basal specific secretions. In such scenario, the coating used to enhance adhesion of the cells to the substrate is also applied on top of the first cell layer after it has adhered to the substrate, thereby acting as a bio-adhesive to ensure proper adherence between the first cell layer and second cell layer. In some embodiments, the bio-adhesive comprises one or more of Matrigel, vitronectin, retronectin, alginate, gelatin, hydrogels. The bio-adhesive between the two cell layers may further include specific growth factors that induce the formation of specific apical and basal interconnections between the two cell layers that further develop into specific neuronal processes and mimic the normal processes of the retina or specific other tissues.

In other embodiments, each cell layer is grown independently on separate substrates. In such embodiments, it may be beneficial to have the second substrate be biodegradable or comprise (or consist essentially of) growth media in a gelatinous state that will dissolve or be degraded and/or absorbed by one or more adjacent cell layers after implantation. The stacked substrates may have a raised perimeter that is at least partially overlapping, thereby protecting the cells sandwiched therebetween and mitigating likelihood of cell migration out of the layer.

The RPE cells used may be of various origins including harvested from the native RPE or iris epithelial cells of eyes or other photo-sensitive organs or tissues, from cell lines derived from native RPE or iris epithelial cells from eyes or other photo-sensitive organs or tissues, from native stem cells derived from embryonic, fetal, or post-natal tissues, or from stem cells derived by any form of reprogramming or directed differentiation (e.g. induced pluripotent stem cells) of mammalian or non-mammalian cells or tissues. The combination of the matrix and first cell layer mimics the organization of the RPE cell-Bruch's membrane complex of the mammalian eye and which is crucial for photoreceptor cell survival and visual function. In a preferred embodiment the matrix is comprised of Parylene C and the RPE cell layer is derived from human stem cells.

Similarly, the photo-sensitive cells (e.g. photoreceptor cells) may be of various origins including harvested from the native photo-sensitive cells of eyes or other photo-sensitive organs or tissues, from cell lines derived from native photo-sensitive cells from eyes or other photo-sensitive organs or tissues, from native stem cells derived from embryonic, fetal, or post-natal tissues, or from stem cells derived by any form of reprogramming or directed differentiation (e.g. induced pluripotent stem cells) of mammalian or non-mammalian cells or tissues. The combination of the first and second cell layers mimics the organization of the RPE-photoreceptor interface of the mammalian eye and which is crucial for photoreceptor cell survival and visual function. In a preferred embodiment the matrix is comprised of Parylene C, the RPE cell layer is derived from human stem cells, and the photo-sensitive cells are photoreceptor cells or photoreceptor progenitor cells derived from human stem cells. In a preferred embodiment the photo-sensitive cells are comprised of a combination of cells with characteristics of cone photoreceptor cells and rod photoreceptor cells. Other embodiments incorporate photo-sensitive cell layers with characteristics of cone photoreceptor cells or rod photoreceptor cells.

Additional cell layers may be comprised of cells with neuronal (e.g. interneurons (bipolar cells, amacrine cells, ganglion cells), glial (e.g. Muller cells, astrocytes), fibroblastic or other attributes beneficial to the structure and function of the tissue complex formed by the first and second cell layers. Additional cell layers may be comprised of relatively pure cell types or mixtures of such in varying ratios. In a preferred embodiment additional cell layers would provide for neuronal connections with photo-sensitive cells of the second layer and with neuronal cells of additional apposed cell layers and/or recipient tissues, organs, organisms. Such organization will mimic the neuronal connectivity of the eye and provide opportunity for neuronal connection of implanted cells/tissue with the nervous system of the recipient, potentiating the likelihood of enhanced photo/visual sensitivity in the recipient.

In additional embodiments the composition of the first, second and any additional cell layers may be reversed or otherwise organized so as to provide structural and/or functional advantages and facilitate alternative means of transplantation.

In additional embodiments, pre-formed tissues are apposed to the basal matrix that provides structural and adhesive support and may also provide molecular cues supporting differentiation and function of the apposed tissue. The pre-formed tissues may be sourced as native tissues from donors or from in vitro differentiation of cells or tissues sourced from cell lines, from native stem cells, or from stem cells derived by any form of reprogramming or directed differentiation (e.g. induced pluripotent stem cells) of mammalian or non-mammalian cells or tissues (e.g. organoids and other three-dimensional cell/tissue cultures).

In additional embodiments, molecular factors are interposed between the basal matrix and the first cell layer, and/or between subsequently apposed cell layers or tissues. Such molecular factors serve to promote adhesion between layers, to enhance structural integrity of the combination tissue, and/or to promote or direct cell/tissue differentiation. Such molecular factors are selected from the proteoglycans, polysaccharides, collagens, laminins, elastins, nectins (e.g. fibronectin, vitronectin, retronectin), minerals, ions, and other soluble or insoluble molecular components of the extracellular or interstitial space. In a preferred embodiment, vitronectin is interposed between the matrix layer and the first cell layer, and interphotoreceptor binding protein is interposed between the first and second cell layers. A natural extract of synthetic formulation of the RPE-Bruch's membrane basement membrane/basal lamina complex can be alternatively interposed between the matrix and the first cell layer mimicking the normal composition of the RPE-Bruch's membrane interface. A natural extract or synthetic formulation of interphotoreceptor fluid and/or matrix material can alternatively be interposed between the first and second cell layers mimicking the normal microenvironment of the photoreceptor-RPE interface in the eye. In alternative embodiments, natural and/or synthetic matrix materials (e.g. parylene or examples from above list of matrix materials), alone or in combination, could be also be interposed between the matrix and the first cell layer, or between the first and second cell layers, and or between any additionally apposed cell layers.

### Cryopreservation Optimization of the Substrate

Although fresh non-cryopreserved cell-based therapies are ideal as no validation and reproducibility of the cryopreserving process and thawing process would be required, from a cost and logistics perspective it is nearly impossible to implement past an academic or small clinical trial.

Controlling nucleation, the onset of change of state from liquid to crystalline, and the temperature compensation provided during controlled-rate preservation for release of latent heat are known to improve post-cryopreservation and thawing cell viability. In various embodiments, the substrate is oriented parallel to the seeded cells due to the configuration and seeding area of the substrate. Therefore, the substrate is in intimate contact with and/or close proximity to all of the seeded cells, thereby allowing for a homogeneous nucleation of all cells simultaneously. During the cryopreservation process, the substrate efficiently induces nucleation without requiring other methods known in the art such as seeding ice crystals or other nucleating agents, mechanical vibration, electrofreezing, etc., which may negatively affect the uniform cell layer formed on the substrate. The substrate configuration and relation to the cells thereby additionally contribute beneficially to the viability of cryopreserved cells in addition to the temperature compensation provided by controlled rate freezing during the process of latent heat release by the seeded cells. The latent heat release is partially dependent on cell lines, but primarily dependent upon the composition of the cryopreservation media used.

Substrates may also have beneficial characteristics such as those seen in the substrate described in U.S. Pat. No. 8,808,687, which is incorporated herein by reference. Substrate characteristics may include a smooth cell growth surface to promote the generation of a monolayer of cells, a perimeter that prohibits cell growth (e.g., perimeter portion of the substrate does not have a thinned membrane portion for sufficient nutrient and waste transport for cell growth affinity, perimeter has a raised lip, etc.)

In specific embodiments, the use of two or more membranes in a sandwich configuration acts as a heat conductive member to accelerate even temperature changes to improve cell viability during the cryopreservation process and the thaw process.

In certain embodiments, the substrate is designed to have an optimal non-planar normal state that conforms to the desired implantation site. Although the substrate may be manipulated during culture and cryopreservation to maintain a planar shape for easier handling and improved cell viability, it may be beneficial for the substrate to have a non-planar shape once implanted. In embodiments where the cell-seeded substrates contain RPE cells and are implanted to adequately cover geographic atrophy areas within the retina, the substrate is optimally curved to match the radial curvature of the retina within the eye. The curvature induces parallel growth of the external limiting membrane (ELM), which is indicative of restoration of photoreceptor microstructures and adjacent visual functionality. Comparatively, RPE cell injections of standalone cells, non-distinct shape cellular gels and suspensions have shown poor clinical outcomes.

The cell-seeded substrate may alternatively be preserved by alternate methods including hibernation, dehydration, or in two or more cell-seeded substrates to be stacked either during implantation, or just prior to implantation.

### Improvement and Increasing Efficiency and Yield Through Manufacturing Steps

With cellular products it is difficult to reproducibly manufacture large quantities of cell-based products on a commercialization scale. Many known laboratory and academic based methodologies have low yields and are difficult to reproduce as they rely heavily on steps that are highly user dependent. Below are examples of some steps to simplify and/or automate some steps to reduce the variability between users to increase manufacturing yield.

Preparation of wells or other surfaces for substrate placement greatly improve yield by reducing the number of touch/transfer steps of the delicate substrates that are less than 100 microns thick and ideally less than 10 microns thick. Such steps include cleaning of the cell plates (i.e. standard 48 well plates used commonly for cellular culturing), placement of the membranes within the wells and reversibly adhering the membranes into the wells by methods of a wet/dry cycle. This reproducible temporary fixation of the membranes onto a culture surface increases yield by reducing loss due to displacement of the substrate during transportation, sterilization, and other subsequent steps. Additionally, this temporary fixation of the membranes onto a culture surface allows for repeated identical placement of the membrane, thereby making it easier for automated cell-seeding steps to accurately follow an efficient seeding pattern, introduction and removal of various fluids to reproducibly flow to targeted areas that do not negatively affect the cell-seeded substrate, and automated peeling off the substrate from the culture surface thereafter.

Bioprinters or specialized machines capable of precise and accurate dispensing of various materials would greatly increase yields. The bioprinters can dispense cells in a specific pattern or interval, thereby seeding the cells onto the substrate to minimize time required for culture to reach specific cellular characteristics (see cell biological evaluation details below for specific cell culture and cryopreservation viability characteristics). For example, the ideal cell seeding density of monolayer RPE cells on a substrate is between 2.0×10⁵ and 7.0×10⁵ cells per milliliter of cell suspension, or between 1.0×10³ and 4.0×10³ cells per square centimeter of substrate surface, or between 1.0×10⁵ and 3.5×10⁵ cells per well of a standard 48-well cell culture plate. A bioprinter can accurately seed the monolayer of RPE cells at a specific density, therefore controllably and reproducibly selecting the number of hours of culture required to reach a specific cell density % (90%-99% subconflucence) whereas when confluence is reached the cell seeding density is closer to 1.0×10⁶ as is understood from standard characteristic growth pattern of cultured cells that follow a log phase growth as cells proliferate. Bioprinters can further be used for accurately placing a bio-adhesive layer onto the substrate, or one or more additional layers of cells grown on a substrate. Bioprinters further contain cassettes or bio-ink cartridges that are easily traceable via lots to allow for cell batch identification.

Large batch culturing of cells for each cell layer is also optimized by having the substrate fixated in identical position and orientation onto the culture surface as the cells can be simultaneously cultured and screened for uniformity prior to seeding onto the substrate or as a secondary or tertiary layer.

In one embodiment, retinal organoids are cultured for 30 to 90 days, generating tissue with bright stratified, laminar layering and organization. Organoids at this stage contain retinal progenitors expressing specific gene expression profiles. In some cases, organoids contain retinal ganglion cells, located centrally with neurites extending throughout the tissue. In some cases, organoids contain a presumptive inner and outer plexiform layer expressing; (protein expression profiles).

Organoids are dissociated into single cells in this time period using; Trypsin or Collagenase or Accutase or EDTA or Papain. Single cells solutions are cultured atop RPE parylene composites using bio adhesives or click antibody chemistry or extra cellular matrix protein mixtures including (list of IPM components), as connective sources generating co-culture multilayered composite implants (see above for various combinations). In some cases, retinal ganglion cell progenitors or immature retinal ganglion cells or mature retinal ganglion cells are isolated using magnetic activated cell sorting (MACS) or fluorescence activated cell sorting (FACS) using markers including but not limited to; THY1 or Brn3a/b or RBPMS. In some cases, retinal progenitor cells are isolated using magnetic activated cell sorting (MACS) or fluorescence activated cell sorting (FACS) using markers including but not limited to; CD37 or (other surface progenitor markers).

Organoid sheets are prepared in this time period by slicing whole tissue into micron thickness sheets using micromanipulator tools such as a scalpel or razor blade. Sheets are affixed to RPE parylene composites using bio adhesives or click antibody chemistry or extra cellular matrix protein mixtures as connective sources generating co-culture multilayered composite implants.

In an additional embodiment, retinal organoids are cultured for 90 to 140 days, generating tissue with bright stratified, laminar layering and organization. Organoids at this stage contain; retinal progenitors (gene expression profiles) and photoreceptor progenitors (gene expression profiles) and maturing photoreceptors (gene expression profiles), maturing interneuron progenitors (gene expression profiles) and amacrine cells (gene expression profiles) and maturing retinal ganglion cells or mature retinal ganglion cells (gene expression profiles).

Organoids are dissociated into single cells using in this time period using; Trypsin or Collagenase or Accutase or Papain. Single cells solutions are cultured atop RPE parylene composites using bio adhesives or click antibody chemistry or extra cellular matrix protein mixtures including; (list of IPM components), as connective sources generating co-culture multilayered composite implants. In some cases, retinal ganglion cell progenitors or immature retinal ganglion cells or mature retinal ganglion cells are isolated using magnetic activated cell sorting (MACS) or fluorescence activated cell sorting (FACS) using markers including but not limited to; THY1 or Brn3a/b or RBPMS. In some cases, retinal progenitor cells are isolated using magnetic activated cell sorting (MACS) or fluorescence activated cell sorting (FACS) using markers including but not limited to; CD37 or (other surface progenitor markers).

In an additional embodiment, retinal organoids are culture for 140-250 days, generating tissue with bright stratified, laminar layering and organoids. Organoids at this stage possess; immature photoreceptor outer segments or maturing photoreceptor outer segments or mature photoreceptor outer segments, apical to the outer layer of the organoids in proximity to the nascent or maturing or mature photoreceptors. Organoids laterally possess retinal interneurons including in some cases; rod bipolar cells and on/off bipolar cells and horizontal cells and starburst amacrine cells. In some cases, organoids possess mature retinal ganglion cells with extended neurites or fragmented neurites.

### Manufacture of a Multi-Cell Type, Multicell-Layered Cell-Based Therapeutic Product

Many ocular diseases such as age-related macular degeneration (AMD) and retinitis pigmentosa require replacement of two or more cell types that make up the stratified layers of a viable retina. According to the progression of a specific disease and size of geographic atrophy, certain peripheral cells of geographic atrophy or layers such as photoreceptors and RPE cells may be rescuable by an implantable cell-based therapy. Thus for optimal improvement, the cell-based therapeutic product should be tailored to specifically replace and integrate into the disease destroyed area, thereby creating a patient specific solution. For example, ideal placement to treat geographic atrophy would include the entire area of geographic atrophy and significant additional area in the “disease transition zone” where RPE and retinal cells are becoming compromised and undergoing death and degeneration and additional peri-lesional area where cells are not yet compromised by the disease process. Alternatively, the implant will be placed over extra-foveal lesions before macular/foveal vision has been impaired with the intent of halting/slowing progression of these lesions that would normally coalesce to form a larger foveal geographic atrophy lesion. In alternative embodiments, the cell-based therapy implants are custom-sized and shaped to specifically cover lesions and adjacent areas holding rescuable cells/tissue.

Below are some example treatment options based on the disease being treated, along with considerations that allow for proper integration with remaining host tissue and regeneration and maintenance of cellular interaction to regain function.

In some embodiments, the cell-based therapeutic product is comprised of a basal substrate or matrix, a first cell layer apposed to the basal matrix, and one or more additional cell layers apposed to the first cell layer. The additional cell layers are in a specific sequence and each additional cell layer may consist of a single cell type monolayer, multi-cell type monolayer, or single cell type bilayer. The basal substrate provides structural and adhesive support for the first cell layer and may also provide molecular cues supporting differentiation and function of the first cell layer. The first cell layer provides structural and adhesive support for the second cell layer and may also provide molecular cues supporting differentiation and function of the second cell layer. Similar attributes of the second and any subsequently apposed cell layers provide similar means of support for any subsequently apposed cell layers. The combination of matrix and multiple cell layers provides for structural and functional attributes that are not attainable by any of the cell layers alone or in combination with any matrix and mimics the organization of ocular cells and tissues.

The basal substrate and each cell layer is optionally coated with a specific bioadhesive to promote proper adhesion to the subsequent cell layer. In some embodiments, the bio-adhesive comprises one or more of Matrigel, vitronectin, retronectin, alginate, gelatin, hydrogels, laminin, collogen, fibronectin, hyaluronic acid, heparin sulfate, methyl cellulose. The bio-adhesive between the two cell layers may further include specific growth factors that induce the formation of specific apical and basal interconnections between the two cell layers that further develop into specific neuronal processes and mimic the normal processes of the cell layers within the retina or specific other tissues. Other molecular cues include those inducing differentiation of the cells, preventing differentiation of cells, maturation of the cells, polarization of cells, improvement of cell junction stability, and formation of organelles for pigmentation, or formation of only a monolayer. In another embodiment, the bio-adhesive consists of two different biotinylated antibodies against cell surface proteins and streptavidin to link them together. The use of anti-body bio-adhesives can beneficially create a temporary adhesion between layers, while not triggering the development of normal processes found within the natural retinal layers.

In one embodiment, the cell-based therapeutic product is specifically tailored to treat geographic atrophy. The basal substrate is comprised of Parylene C, the first cell layer is comprised of retinal pigmented epithelial (RPE) cells, and the second cell layer is comprised of photoreceptor cells. This organization mimics that of the organization of the RPE-photoreceptor interface of the mammalian eye that is crucial for photoreceptor cell survival and visual function. The cell-based therapeutic product can be placed to cover the entire geographic atrophy, thereby fully replacing the atrophied cells while making interconnections directly with the patient's remaining cells.

In yet another embodiment, the cell-based therapeutic product is specifically tailored to replace laser induced or physical trauma induced damage beyond the retinal pigmented epithelial cells. The basal substrate is comprised of Parylene C, the first cell layer is comprised of retinal pigmented epithelial (RPE) cells, and the second cell layer is comprised of photoreceptor cells, the third layer is comprised of bipolar cells interspersed with amacrine cells and horizontal cells, the fourth layer is comprised of ganglion cells. This organization mimics that of the organization of the RPE-photoreceptor-optic nerve interface of the mammalian eye that is crucial for visual function. The cell-based therapeutic product can be placed to cover the entire trauma site, thereby fully replacing the full retina tissue to the optic nerve.

In yet another embodiment, the cell-based therapeutic product comprises a second substrate. The first and second substrate sandwiches therebetween at least one cell layer. The sandwich configuration prevents cell dispersion, sloshing off, or unwanted migration of cells during the implantation procedure in which the substrate may be rolled into a surgical instrument.

In certain embodiments in which a larger area of tissue must be replaced, two or more implants may be inserted and oriented parallel to each other. In this case, each substrate may have interconnecting perimeters to promote the connection of the implants on the substrate layer, thereby preventing the mismatching of the cellular layers between each implant. Such modular format of the implants is beneficial in that the surgical access point can remain small (i.e. less than 2 mm), thereby minimizing the occurrence of hemorrhage, sudden intraocular pressure drop, and other adverse events related with larger (greater than 2 mm, and in most cases greater than 3.5 mm surgical access points).

Each batch of cell-based therapeutic product can be tested via batch sampling and destructive and non-destructive testing procedures. Each cellular layer may be visually evaluated for visual characteristics but also tested for expression levels of genes. Specifically, each cell type has a list of cell identity markers that are categorized into 3 ranges of normal as known in literature, non-integrated, and integrated-functional. Each of these cell identity markers are provided specific thresholds to confirm if the cell-based therapeutic product is ready for patient use. RPE cells are tested for one or more of: RPE65, RLBP1, PEDF and S100A4. Photoreceptor/Retinal progenitor cells are tested for one or more of: VSX2 (CHX10), CRX, NTSE (CD73), PAX6, RAX, SIX3, SIX6, LHX2 expression. Rod Photoreceptors are tested for one or more of NRL, SAG, RHO, GNAT1, GNGT1, and PDE6G. Cone Photoreceptors are tested for one or more of OPNISW, OPN1MW, OPN1LW, ARR3, PDE6H, GNGT2, and GUCA1C. Bipolar Cells are tested for GRIK1, PRKCA, VSX1, VSX2, CHTF18, TMEM215, LRTM1, IRX6, PCP2, and TRPM1. Retinal Ganglion Cells (RGC) are tested for SLC17A6, RBPMS, SNCG, SYT2, NEFL, THY1, NEFM, and POU4F2 (BRN3B). Muller Glia are tested for GLUL, HES1, HES5, GFAP, CRALBP, APOE, RLBP1, and CRYAB. Pericytes/Endothelial cells are tested for COL1A2, ACTA2, PDGFRB, VWF, TEK (TIE2), NOS3 (ENOS), and PECAM1 (CD31).

Additional evaluation may be completed by immunostaining to confirm apical markers and basal markers, quantification of markers by fluorescence. The multi-layered product may also be physically manipulated through wash steps, additional culture, and handling with a specialized tool to physically test the binding affinity o the layers.

Yet additional evaluations may be completed by metabolic testing including but not limited to mitochondrial respiration, metabolic NADPH turnover for organoids, and responses to light or polarizing pulses.

Testing of proper integration into the patient's preexisting tissue can be done by conventional ophthalmic testing including optical coherence tomography (OCT) and more specialized testing such as superior colliculus electrophysiology testing, optokinetic testing.

### Cryopreservation Optimization: Cell Biological Evaluation

In embodiments involving cryopreserving cells seeded on a substrate, optimal cryopreservation viability characteristics may include non-polarized or partially polarized cells, and/or cells in a subconfluent monolayer configuration. In embodiments that involve cryopreserving RPE cells seeded on a substrate, the RPE cells may be selected and cryopreserved when representing optimal cryopreservation viability characteristics. Optimal cryopreservation viability characteristics may include (i) non-pigmented RPE cells (including depigmented RPE cells), (ii) non-polarized or partially polarized RPE cells, (iii) RPE cells without mature cobblestone morphology, (iv) RPE cells with gene expression levels below that of mature cells or lacking specific gene expression, (v) RPE cells in a subconfluent monolayer configuration, or various combinations thereof.

One characteristic of RPE cells exhibiting optimal cryopreservation viability is non-pigmentation or limited pigmentation. RPE cells contain numerous melanosomes, i.e., pigment granules that extend from the apical area into the middle portion of the cell. Certain RPE cells such as those interfacing with the macular area are more densely pigmented. Non-pigmented or limited-pigmentation RPE cells can be non-fully differentiated, non-isolated, and/or non-purified cells. Various methods may be implemented including depigmenting RPE cells by chemical removal (e.g., melanogenesis inhibitors), alteration of growth media, interaction with physiologically adjacent cell types, etc.

Another characteristic of RPE cells having optimal cryopreservation viability is nonpolarization or partial polarization. Polarized RPE cells distinguish between the apical (corresponding to the retinal facing side of the RPE cells) and basal (corresponding to the choroidal facing side of the RPE cells) directions that mimic physiological characteristics including apical microvilli, well-defined tight junctions, membrane transport capability and melanocytic pigmentation.

Yet another characteristic of RPE cells exhibiting optimal cryopreservation viability is the lack of a cobblestone morphology. RPE cells of a differentiated, isolated, and/or purified cells take on an appearance of cuboidal cobblestone morphology.

An additional characteristic of RPE cells having optimal cryopreservation viability is gene expression levels below that of mature cells or lacking specific gene expression. This characteristic may be tested for by RNA or other nucleic acid expression, protein expression, lipid expression, glycosylation pattern, immunohistological staining, or electrophysiological properties. Secondary measurement techniques such as measurement of secretome levels may also be alternatively or additionally implemented.

Another characteristic of RPE cells having optimal cryopreservation viability is a subconfluent monolayer configuration. Confluence is achieved when cells fully grow into all available portions and reach contact inhibition. After reaching confluency, many cell types including mammalian cell types exhibit different characteristics compared to sub-confluency. The ideal cell seeding density of monolayer RPE cells on a substrate is between 2.0×10⁵ and 7.0×10⁵ cells per milliliter of cell suspension, or between 1.0×10³ and 4.0×10³ cells per square centimeter of substrate surface, or between 1.0×10⁵ and 3.5×10⁵ cells per well of a standard 48-well cell culture plate. Once confluence is reached the cell seeding density is closer to 1.0×10⁶ as is understood from standard characteristic growth pattern of cultured cells that follow a log phase growth as cells proliferate. Cell density can be measured by image analysis, spectrophotometry, electrical/impedance analysis along with other more invasive/destructive processes. A monolayer, compared to a multi-cell layer, is promoted by a flat cell-seeding surface of the substrate and matching the substrate nutrient/waste transport ratio to support a monolayer.

Optimal cryopreservation viability of RPE cells is achieved from three to 10 days following seeding of the RPE cells on the substrate, at which point the cells have not reached confluence and fully differentiated on the substrate. RPE cells with optimal cryopreservation viability are obtained during this time window, but may vary according to the cell line used, growth media, substrate characteristics, etc. Therefore, one or more of the cell characteristics described above as associated with optimal cryopreservation viability should be reviewed both qualitatively and quantitatively prior to the cryopreservation of the cell-seeded substrate. The above-mentioned substrate and cell line characteristics should be taken into account to create optimal cryopreservation and thawing protocols to maximize cell viability and functionality and substrate integrity.

### Optimized Cryopreservation Protocol and Considerations: Cryo-Hybernation

Cryo-hibernation protocols, an alternative to standard cryopreservation protocols, have shown increased viability of thawed cell-seeded on substrates. In various embodiments, a cryo-hibernation protocol is employed as follows. Following the initial controlled-temperature ramp-down phase, once a first temperature below the latent heat release of the seeded cells is reached (between 0° C. and −20° C.), the cells are kept at the first temperature for a first period of time. The first temperature is any temperature below the latent heat release temperature and may be, for example, −20, −30, −40, −50, −60, −70, −80, −90, or −100° C. The first period of time may be, for example, 12 hours, 1 day, 7 days, or 28 days, and helps acclimate the cells to a cryopreserved state without immediate drastic changes to the temperature. This acclimatization additionally prevents microtears from forming in substrates as may be caused by rapid temperature reduction (−1° C./min or greater). After the first period of time, the cryopreserved cells are transferred and maintained at a storage temperature for a second period of time. In most cases, the storage temperature is −196° C. (e.g., the temperature of liquid nitrogen) for convenience, but may be the same temperature as the hibernation temperature.

In certain embodiments, the cells are kept in set temp freezers and carrying cases at the first temperature until thawing. Although this will require a portable set temp freezer, long term hibernation temperatures increases cell viability post-thaw as the thawing process has a smaller temperature differential than that from −196° C. (e.g. temperature of liquid nitrogen), and thereby has significantly less temperature variation zones which causes variable ice crystal formation patterns and differing cryoprotectant removal rates for batch cell-seeded substrate thawing as well as within each individual substrate on a smaller scale.

### Cryopreservation Optimization: RPE Cell Lines

In certain embodiments involving hESC-RPE cells, various combinations of cryopreservation protocols were tested and optimized for the specific cell line. Although tested with a specific cell line, similar outcomes are anticipated for other RPE cell lines as major factors of cell line and substrate characteristics as described above were taken into consideration. The RPE cells of CPCB-RPE1 implants showed optimal cryopreservation viability characteristics including (i) non-pigmented RPE cells (including depigmented RPE cells), (ii) non-polarized or partially polarized RPE cells, (iii) RPE cells without mature cobblestone morphology, (iv) RPE cells with gene expression levels below that of mature cells or lacking specific gene expression, (v) RPE cells in a subconfluent monolayer configuration, or a combination thereof. This combination of cell-optimal cryopreservation viability characteristics was obtained between 3-10 days (7 days being the median day between batches) after seeding onto the implantable substrate. Cryopreservation viability-promoting characteristics were taken into account for substrate design; these included the coefficient of thermal expansion of the substrate, a substrate elasticity parameter, substrate thickness, and substrate implantation size.

The CPCB-RPE1 cell line was tested with various combinations of cryoprotective agents and freezing rates. The most viable combination was the use of CS-10 (manufacturer: BioLife Solutions, Bothell, Washington) having a DMSO concentration of 10%, and freezing between the ranges of −5° C./min and −30° C./min. Comparatively, DMSO concentrations of 2% and 5% as well as freezing rates between −1C and −3C have lower cell viability outcomes.

### Cryopreservation Optimization: Substrate Cryopreservation Device

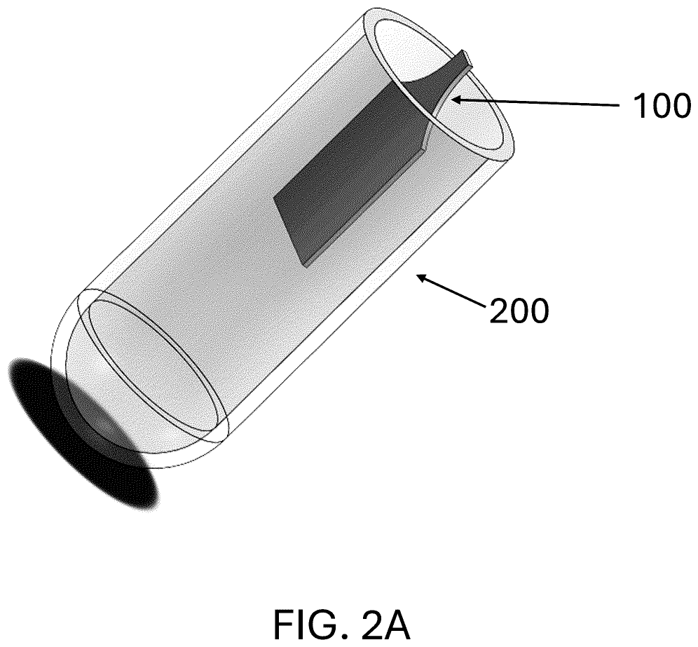

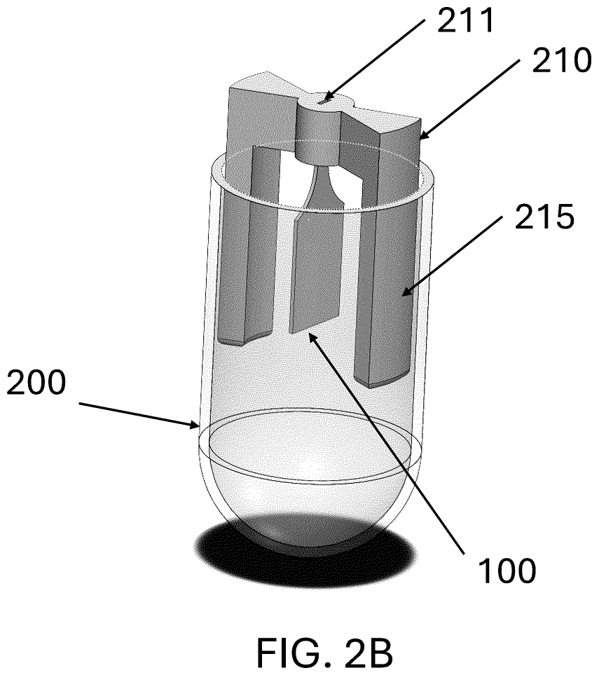

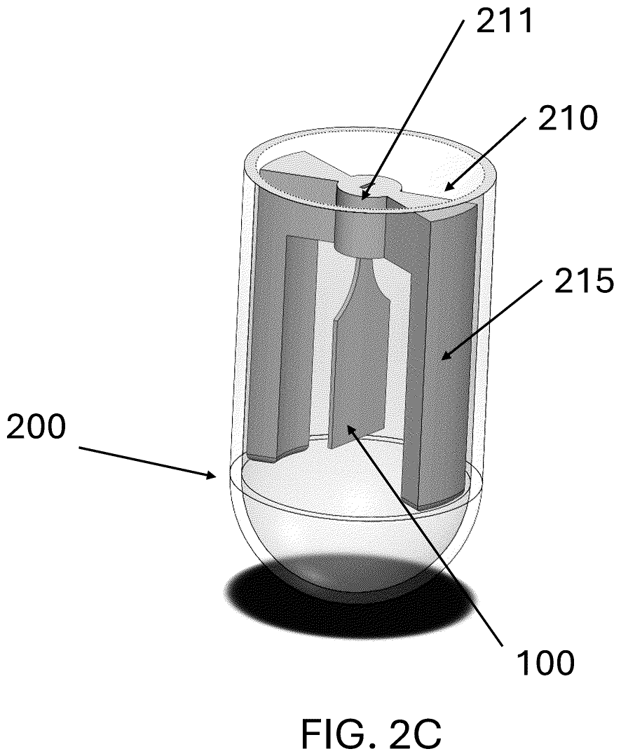

Standard lab materials and protocols designed for cryopreserving and thawing may be detrimental to cell-seeded substrates as they were designed for single-cell or cluster-cell cryopreservation and not for substrate cryopreservation. For example, the standard cryogenic vials and tubes are conical, round or have square bottoms. When a substrate is inserted into the cryovial, even with delicate guiding by the handling tab, the substrate is susceptible to contacting the sides and bottom of the cryovial which may dislodge cells or tear thin substrates. See prior art FIG. 2A. Additionally, during the cryopreservation and thawing processes, the generation of ice crystals in a non-uniform fashion may also dislodge cells or damage the substrate, especially as turbid and sheer stresses are formed through any swirling or agitation which are amplified by the conical shape of tubes.

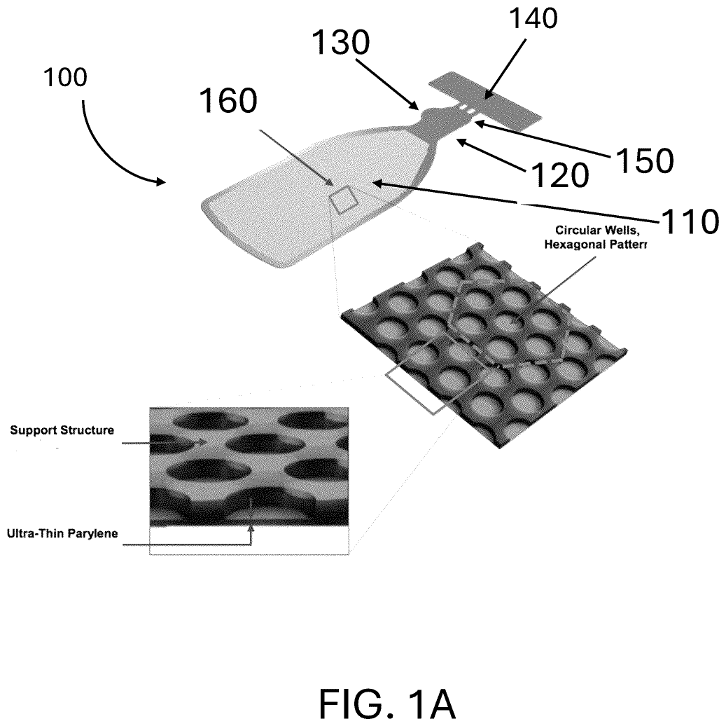

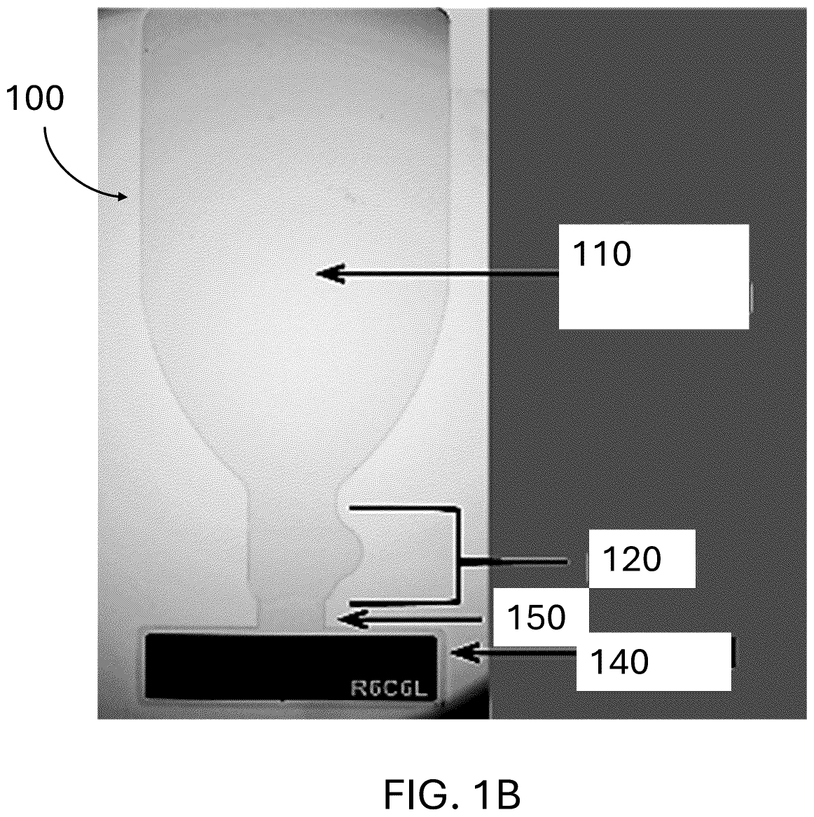

FIG. 1A shows the various components of a substrate 100 (also sometimes referred to herein as implant 100) for cell-seeding including the implant body 110, head 120, optional tab region 150, and handle 140. The implant body 110 includes the ultra-thin membrane layer 160 onto which cells will be seeded. The head 120 portion consists of an orientation bump 130 to identify when the top facing side of the implant body is the ultra-thin membrane layer 160 onto which the cell-seeding will occur (e.g., when the handle is proximal and the implant body is distal, the bump on the right signals that the ultra-thin membrane layer 160 is the top side and the support structure is on the bottom side. The head 120 portion of the substrate is graspable by a surgical instrument, e.g., an instrument as described in U.S. Pat. No. 11,478,272, incorporated herein by reference, after which the surgical instrument extends a tubular sheath over the substrate 100, thereby folding the substrate 100 in a concave shape to protect the cells within during the implantation procedure. The handle 140 is used for handling the substrate 100 with forceps or other instruments during manufacture and subsequent processing steps. The handle 140 may include a titanium or other material embedded within the polymer for increased durability during handling as well as indicia (e.g., a serial number) to uniquely identify the lot and manufacturing information and/or other information of the substrate for traceability. Finally, the optional tab region 150 is where the handle will be removed by a cutting process prior to nestling the folded substrate within the custom surgical instrument. The tab region 150 may be one uniform piece, perforations, or a pre-marked portion to act as a guideline for where the handle should be cut and removed prior to implantation.

FIG. 1A additionally illustrates progressively enlarged views of the substrate's cell-seeding region for use in connection with various embodiments of the present disclosure. This membrane embodiment consists of a support structure layer patterned in a hexagonal pattern for enhanced flexibility and durability and an ultra-thin membrane onto which the cells are seeded. Embodiments employing other patterns including a square pattern, spiral, fret pattern, or various other repeating or self-similar patterns may be alternatively used. Many of the above-mentioned patterns have been tested for durability after repeated flexing and used in the flexible circuit manufacturing industry.

FIG. 1B is the top view of the substrate 100. The implant body 110 is seen with a tapered proximal end which helps induce the implant body 110 to fold into a concave, taco-like shape when the tubular sheath of the custom surgical instrument is extended over the implant body 110. In this embodiment, the optional tab region 150 is one uniform section allowing flexibility in where the handle 140 is removed from the implant body 110. The handle 140 has a biocompatible implant grade titanium piece embedded within the substrate material that is marked by laser etching with an identifiable number.

The creation of a substrate cryopreservation unit is ideal to accommodate the characteristics of the cell-seeded substrate and improve viability of seeded cells and minimize damage to the substrate. Below are a few embodiments of substrate cryopreservation apparatuses.

FIG. 2A is a partially transparent perspective view of a cell-seeded substrate 100 as shown in FIG. 1 disposed within in a standard cryovial 200 with no assisted holder. The substrate 100 may be of various sizes to accommodate the target implantation area. For example, for a retinal implant, the target area of geographic atrophy which should have a majority of the area covered should equal the target size of the substrate 100. The substrate 100 should comfortably fit within the cryovial with ample distance from the sides and bottom of the cryovial to minimize and ideally avoid physical interaction. The cryovial 200 itself is filled with a cryoprotectant media. Various solutions of cryoprotectant media is available and helps protect the cells from intracellular ice formation and lysing. Common cryoprotectant media includes DMSO, glycerol, ethylene glycol, propylene glycol, and other additives such hydroxyethyl-starch, polyvinyl pyrollidone.

The first embodiment shown in FIG. 2B-2C incorporates a substrate holder 210. The bottom of the substrate holder 210 includes a substrate retaining mechanism 211 into which the handle end of the substrate 100 may be inserted. The substrate handle may be manufactured to have an extended or removable portion that may be cut to remove the substrate from the substrate holder. The substrate holder further comprises one or more prongs 215 to limit the ingress distance into the vial, therefore preventing the substrate from contacting the bottom of the cryovial 200. The prongs 215 are parallel to the inner walls of the cryovial to prevent or limit tilting of the substrate 100. The substrate holder 210 may further comprise a latch for pressure based grasping and release of the substrate. The substrate holder may alternatively have other substrate retaining mechanisms 211 including: a vice, friction surface, retaining through rod, etc.

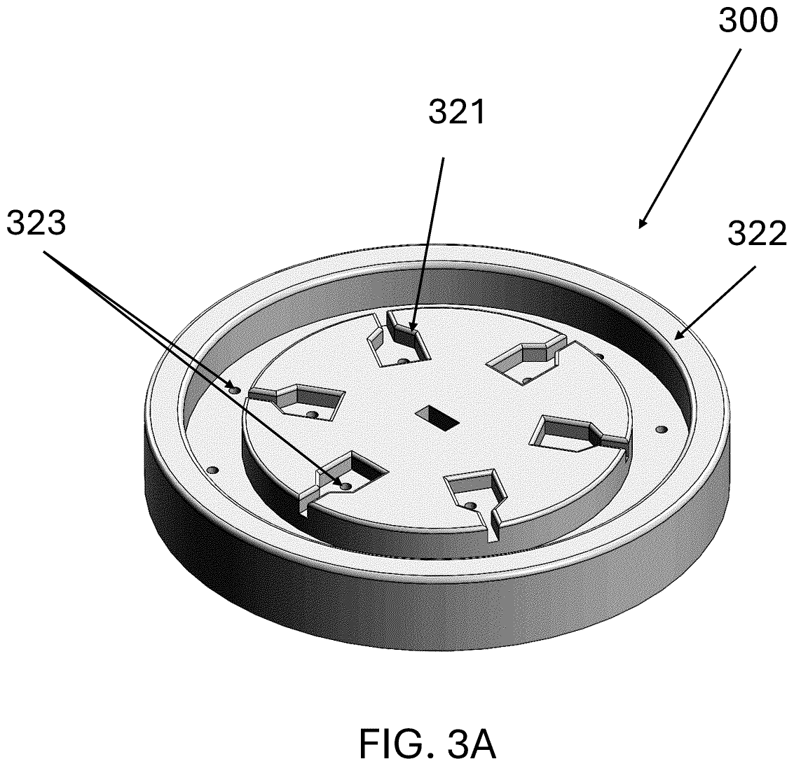

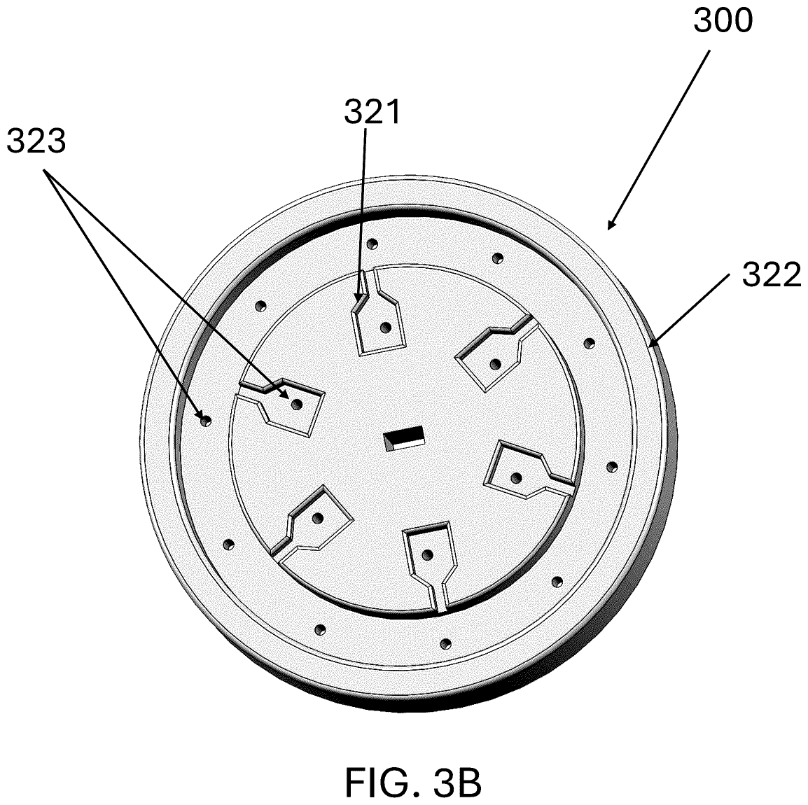

In an alternative embodiment shown in FIGS. 3A-3B, much like a cryopreservation cane that can hold numerous cryopreservation vials, a single cryopreservation tray 300 may house multiple cell-seeded substrates and be cryopreserved at once. Each tray has multiple slots 321 into which an individual substrate can be placed. A retaining perimeter wall 322 allows for a fluid such as a cryoprotectant to be filled to cover each substrate prior to cryopreservation of the whole cryopreservation tray 300. The cryopreservation tray 300 has multiple drainage holes 323 on the bottom surface through which the fluid within the tray can be drained.

An example process of thawing is as follows. The substrate cryopreservation housing is removed from the liquid nitrogen storage tank and placed in a heated circulating water bath or other controlled temperature increasing device until the substrates are thawed and the cryopreservation media is removed by replacement with balanced salt solution or other liquid through a washing process. Once thawed and the cryopreservation media is removed from the substrate, it is ready for implantation.

### Cell-Seeded Substrate Cutting and Loading Pedestal

As described in FIG. 1A, the handle is used for handling the tab with forceps or other instruments during manufacture and subsequent processing steps but must be cut and removed prior to inserting the substrate within the custom surgical instrument for implantation. To ensure a repeatable, uniform, and clean detachment of the removable handle portion and loading of the implant into the custom surgical instrument, a custom loading pedestal and custom holding/cutting tool are desired.

A multi-well plate is commonly used for cell culturing and assays. However, due to the limited base area and high walls of each well, they are not ideal for the handling of cell-seeded substrates that are the size of a majority of the base area of each well. Furthermore, with the membranes laid flat on the bottom of the well, the high walls make it difficult to manipulate the handle or tab portion with tweezers, forceps, or other instruments.

The various embodiments described below of a cutting and loading pedestal aids in preoperative preparation of the substrate that involves rinsing, removing the handle, and loading the ready implant into the custom surgical tool. The main features include variations of a wall dividing the rinse and cutting regions, a ramp for angling the handle of the substrate for easier accessibility, a wall for positioning the membrane for loading, and a circumferential wall that contain the washing solution. Embodiments containing one or more of the above features may make the implant prepping process easier to accomplish.

Pedestals in accordance with certain embodiments may be particularly configured to receive and temporarily hold an implantable substrate (e.g., cell-seeded membrane) during steps to prepare the implantable substrate for implantation. These steps may include, for example, washing and/or cutting of the implant. In some embodiments, the pedestal includes a floor and a ramp nook having one or more sidewalls protruding from the floor. The floor, in some embodiments, may be a generally planar surface and, during use, provides a horizontal working surface on which the implant and/or liquid may be positioned. The one or more sidewalls of the ramp nook may protrude vertically from the planar floor and at least partially define a cavity within the ramp nook that, in some embodiments, is particularly sized and shape to receive at least a portion of the implantable substrate. In some embodiments, the cavity includes a sloped bottom surface that is angled obliquely relative to a plane of the floor. In some embodiments, the sloped bottom surface slopes to a point below a plane of the floor to define a well in the floor. The well may be used to contain liquid, for example, which can help keep the implant moist during handling. In some embodiments, the sloped bottom surface may be configured to hold the implant at an angle relative to the floor such that, for example, a portion of the implant that extends out of the cavity of the ramp nook may be raised above the floor to facilitate handling. In some embodiments, the cavity of the ramp nook may be sized and shaped to receive only a first portion of the implantable substrate (e.g., implant body 110 and/or head 120 of implant 100) while a second portion of the implantable substrate (e.g., handle 140 of implant 100) extends outside of the cavity. The second portion of the implantable substrate may be positioned in a cutting region of the floor outside of the ramp nook, for example, such that the second portion is accessible for cutting.

In further embodiments, the pedestal may include one or more dividing walls that also protrude from the floor. In some such embodiments, a dividing wall may at least partially divide the floor into two or more regions, for example, a cutting region on a first side of the dividing wall and a washing region on a second side of the dividing wall. In some embodiments, the sidewalls of the ramp nook may be extend directly from a side of the dividing wall. Thus, in some embodiments, the dividing wall may also partially define the cavity of the ramp nook. In some embodiments, the sloped bottom surface of the cavity of the ramp nook may slope upwards as the bottom surface extends away from the dividing wall. In yet further embodiments, the pedestal can include a perimeter containment wall that surrounds a portion or all of the floor. In some embodiments, the pedestal is configured to contain a liquid (e.g., rising solution) in an area bounded by the perimeter containment wall. The perimeter containment wall height may be sufficient to contain a volume of liquid to fully submerge the implantable substrate according to some embodiments. The perimeter containment wall may include indicia or markings to identify the volume of liquid contained in the pedestal. The pedestal may be open at top to allow the implant and/or liquid to be placed on the pedestal from above during use. In some embodiments, the floor may include one or more ports that are configured to introduce and/or remove liquid from below through the floor of the pedestal. The ports may be opened during liquid introduction/removal, and closed to retain liquid in the pedestal (e.g., during implant washing) according to some such embodiments.

One or more components of the pedestal may be integrally formed with each other such that they are of a seamless, unitary construction. For example, the floor, ramp nook (including side walls), dividing wall, and/or perimeter containment wall may be integrally formed from a common material (e.g., plastic). For example, the components of the pedestal may be integrally molded from a plastic material. In some embodiments, components of the pedestal may be formed by an additive manufacturing process (e.g., 3D printing).

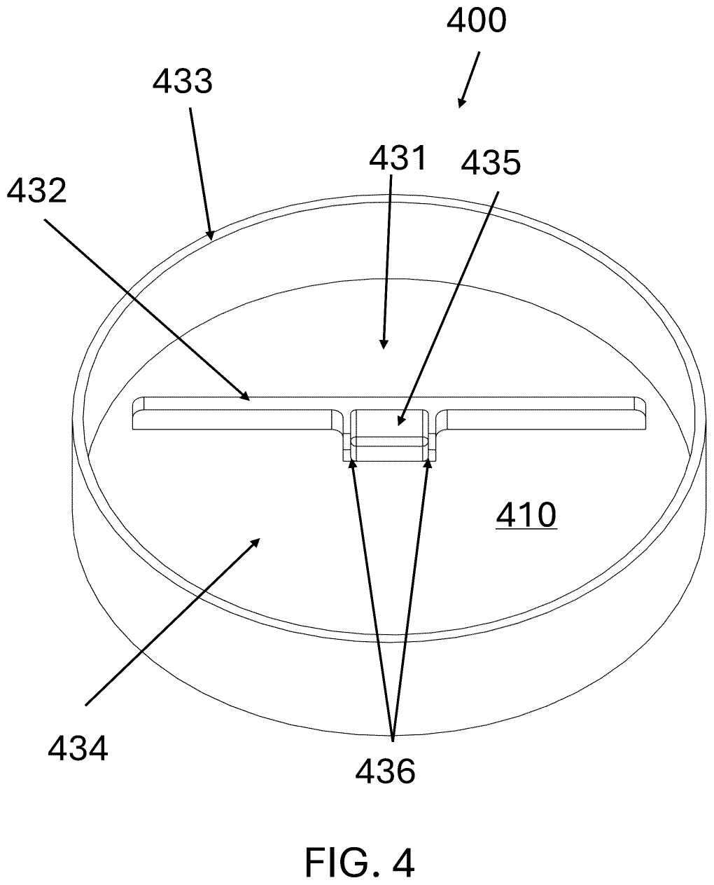

FIG. 4 shows an example pedestal 400 according to certain embodiments of the present disclosure. Pedestal 400 may be used in preparing an implantable substrate (e.g., implant 100) in which the substrate is washed to remove cryoprotectant (e.g. dimethyl sulfoxide (DMSO)) post cryopreservation thaw, cut and remove the handle, and load the implant into the custom surgical tool. In some embodiments, pedestal 400 includes a washing region for rising/washing the cryoprotectant and/or other substances from the implant, and a cutting region configured to receive the implant to facilitate cutting of the implant. In some embodiments, pedestal 400 includes a ramp nook 435 which includes one or more raised walls (e.g., sidewalls 436) that at least partially defines a space that is sized and shaped to receive at least a portion of the implant (e.g., implant 100). The space defined by ramp nook 435 may have a shape that matches a shape of implant 100 (e.g., the shape of implant body 110 and/or head 120). In some embodiments, handle 140 is not received within ramp nook 435, such that handle 140 may remain exposed for cutting and removal. In some embodiments, ramp nook 435 defines a well or cavity in the floor 410 of pedestal 400 for receiving implant 100. The floor 410 of pedestal 400 may refer to a bottom inner surface of pedestal 400, which may be planar and generally oriented horizontally during typical use. In some embodiments, the well or cavity defined by ramp nook 435 may also be configured to contain an amount liquid (e.g., buffer, salt solution) for keeping the implant moist during handling. In some embodiments, the well or cavity include a bottom surface that is angled obliquely relative to the floor of pedestal 400. In some embodiments, ramp nook 435 is located in a central portion of pedestal 400, spaced away from a preriphery of pedestal 400. In some embodiments, ramp nook 435 is located in the cutting region of the pedestal 400. In some embodiments, ramp nook 435 may extend from one side of a wall that protrudes from the floor of pedestal 400 (e.g., dividing wall 432). In some embodiments, the bottom surface of ramp nook 435 may be sloped upwards as it extends away from the wall.

In some embodiments, use of the pedestal 400 can include use of the following components and steps:

Preparation Step 1: A sterile field workstation is setup to process the implant (e.g., cell-seeded substrate). This may be in the operating room or an adjacent room from which the prepared implant can be quickly moved thereafter for implantation.

Preparation Step 2: The cryoshipper or other cryopreservation carrying unit is opened and one vial or other container containing one or more cryopreserved cell-seeded substrates are removed and thawed in a cryovial block, or similar container which thaws the cryopreserved cell-seeded substrate by either room temperature or controlled heating mechanism. Once thawed, the cryovial or container is opened and the implant is retrieved using the custom forceps for the implant or standard fine tip forceps.

Step 1: Implant Rinsing/Implant Rinsing Region. The upper part 431 of the pedestal 400, above the dividing wall 432 looking at it from a top view is the region for rinsing the implant. Here, the implant is submerged in, e.g., balanced salt solution (BSS) immediately after thawing to remove cryoprotectant from the seeded cells. While still submerged, the implant is then moved over to the cutting region 434.

Step 2: Cutting Handle/Implant Cutting Region. The cutting region 434 of the pedestal 400, below the dividing wall 432 in FIG. 4, is the region where the handle of the implant (e.g., handle 140 of implant 100) is cut off the membrane. The implant handle portion may be cut with a standard scalpel or, in some embodiments, by using a specialized cutting tool as described further below. While still submerged is the BSS, the implant is moved to the ramp nook 435 for loading.

Step 3: Loading Implant/Ramped Nook Region. This region of the dividing wall 432 serves two functions: to maintain the continuity of the dividing wall 432 and to serve as a backdrop for the ramp nook 435 to contain the implant in the ramp region during loading. This ramped nook 435 is angled downward compared to the rest of the pedestal inner floor. The implant is pushed into the ramped nook 435 implant body first, thereby lifting the handle 140 of the implant slightly upward making it easier to grasp with a specialized instrument (e.g., forceps).

The pedestal 400 additionally includes a dividing wall 432 which serves as a landmark that divides the rinsing region 431 and the cutting region 434. Dividing wall 432 also serves as a guide towards the ramp region 435 should the implant 100 travel unexpectedly to another region of the pedestal 400 (e.g., by movement of the rising solution). The cutting region 434 or floor of the pedestal 400 on which the implant 100 will have the handle 140 cut away, the floor may optionally be reinforced with an embedded layer of medical grade titanium or other material that will prevent production of unwanted debris.

The ramped nook region 435 additionally includes small filleted side walls 436 of the ramp nook 435 positioned at an appropriate distance apart to accommodate the width size of the implant, thereby correctly orienting the implant for easy access and loading into the custom surgical tool.

The pedestal additionally includes a perimeter containment wall 433 to contain sufficient rinsing fluid (e.g. balanced salt solution, buffer solution, or other wash solution) to completely submerge the implant for the rinsing process. Perimeter containment wall 433 may surround floor 410 such that the rising fluid is contained on floor 410 of pedestal 400 during rising in a volume sufficient to at least submerge the implant. The perimeter containment wall 433 may have notches or other readable markers or indicia identifying the volume of fluid contained in the pedestal, thereby showing when sufficient fluid to submerge the implant has been added.

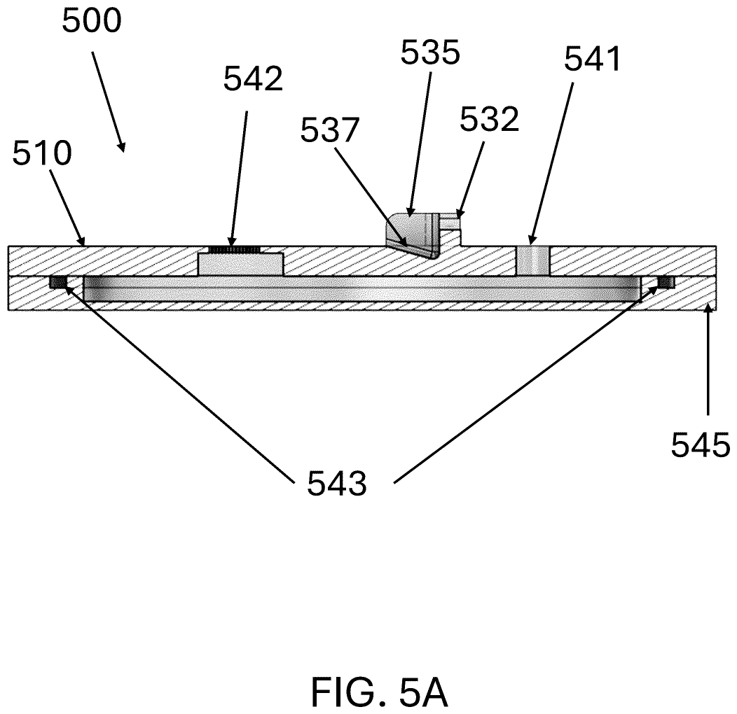

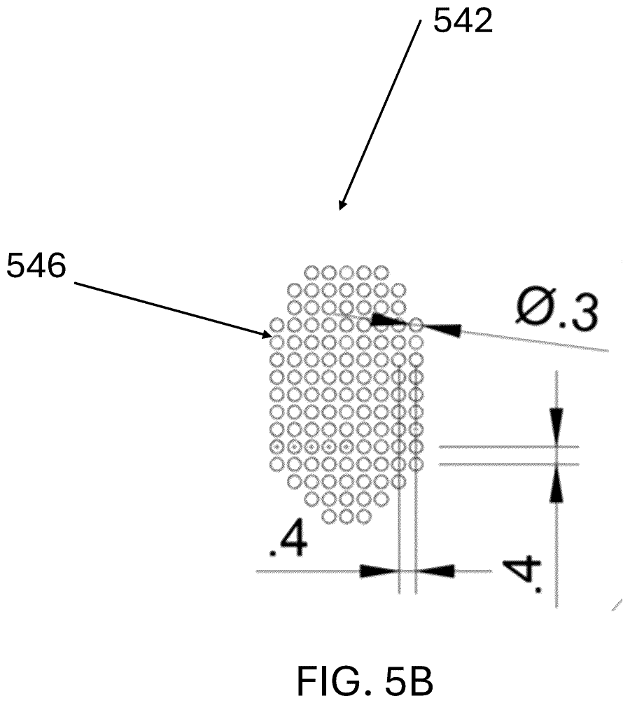

FIG. 5A shows a pedestal 500 according to a further embodiment. Pedestal 500 includes a ramp nook 535, which may be similar to the structure described for ramp nook 435. In some embodiments, ramp nook 535 includes a sloping bottom surface 537 that slopes to a point below floor 510 of pedestal 500. Pedestal 500 may also include a dividing wall 532 that is similar to dividing wall 432. In some embodiments, the pedestal 500 contains one or more inlets and outlet ports 541 on the bottom surface (floor of the pedestal). The inlet and outlet ports 541 are opened and closed independently to add or remove liquid reagents such as the balanced salt solution, thereby allowing the replacement of solution during the washing process and to remove the washing solution once the washing step is complete. The ports 541 are ideally small enough and optionally covered with a sieve 542 for the washing solution to flow through, but small enough that no part of the implant can ever be caught in any port. By having numerous ports 541, it also prevents any rotational currents or whirlpooling effects that may cause sheer forces to damage the membrane or remove the seeded cells. The ports 541 may be attached to a positive pressure pump for filling fluid or a vacuum pump for removing fluid. FIG. 5A depicts an embodiment of the ports 541 including the sieve structure 542 of the ports 541. By having the ports 541 on the bottom surface of the pedestal 500, the fluid ebbs and flows in from below, thereby mitigating risk of a fluid applied from above washing about the cells seeded on the top surface of the implant 100. The washing fluid may be held below the floor 510 of pedestal 500 and there may be a separate fluid exchange apparatus layer 545 which is aligned to the pedestal by one or more stack alignment pins 543. While not shown in FIG. 5A, some embodiments of pedestal 500 may also include a perimeter containment wall that surrounds floor 510 in a manner similar to perimeter containment wall 433.

FIG. 5B shows an embodiment of a sieve 542 with a repeated pattern of holes 546 that are proportionally smaller than the implant 100 as a whole.

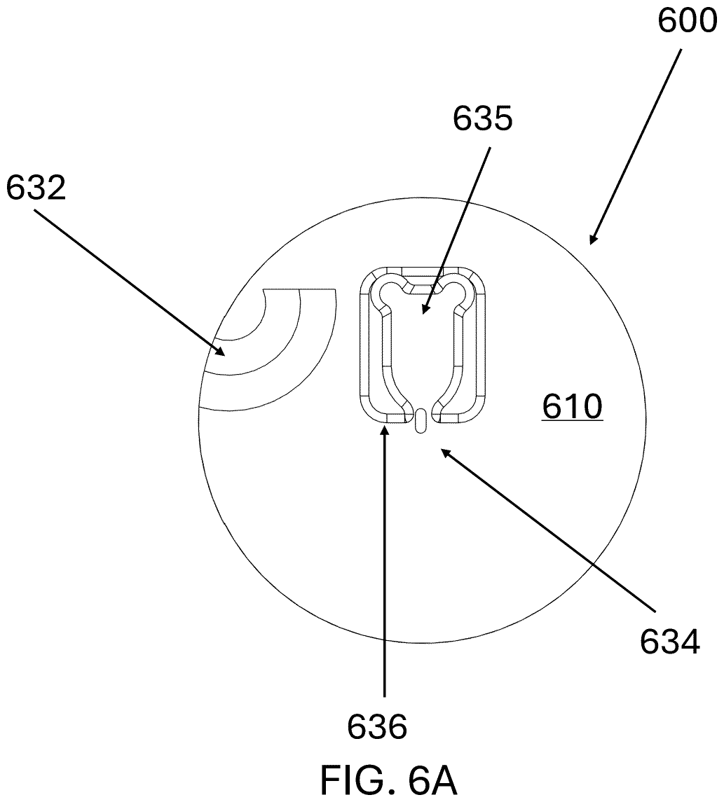

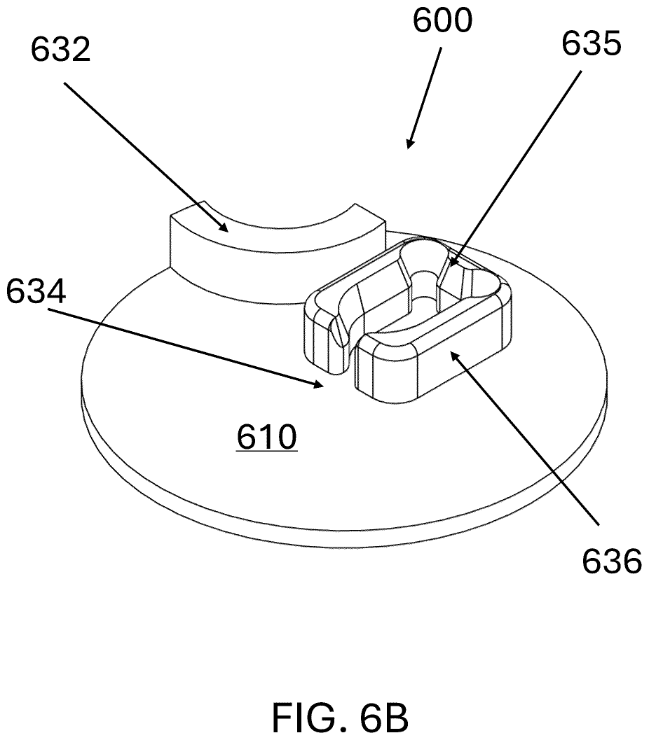

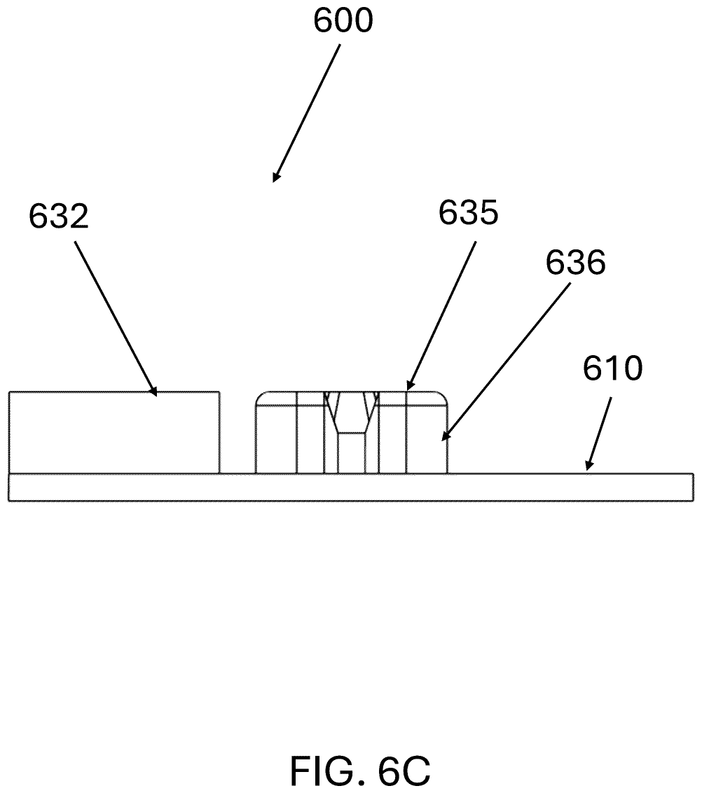

FIG. 6A-6C shows an alternative embodiment of a pedestal 600. The ramp nook 635, which protrudes from floor 610, defines a cavity that is specifically shaped to the implant and is ideally a proportional size larger (e.g. 110%, 120%, 130%, etc.) to allow for easy placement of the implant into the ramp nook. The side walls 636 of the ramp nook may have an inner surface shape and outer surface shape that are different. In some embodiments, the rinsing can occur in the cavity of the ramp nook 635. The implant cutting region 634 is just outside of the ramp nook 635. This embodiment still has one or more dividing walls 632 to control fluid motion in the pedestal 600.

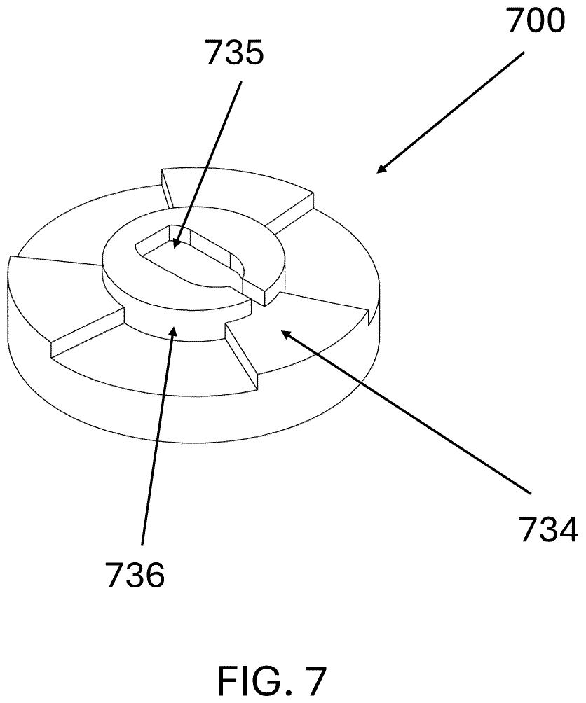

FIG. 7 shows yet another embodiment of the implant cutting region 700 that may be used with a pedestal (e.g., pedestals 400, 500, 600) which is shaped to be the size and partial shape of the implant and is ideally a proportional size larger (e.g. 110%, 120%, 130%, etc.) to allow for easy placement of the implant into the ramp nook 735. By placing the implant in the ramp nook 735 with the handle placed in the cutting region 734, the cut is always uniform and reproducible. A circular cut is made by aligning a circular trephine blade's inner bore with the side walls 736 of the ramp nook 735 and making a push down or twisting motion to cut the handle portion of the implant for removal.

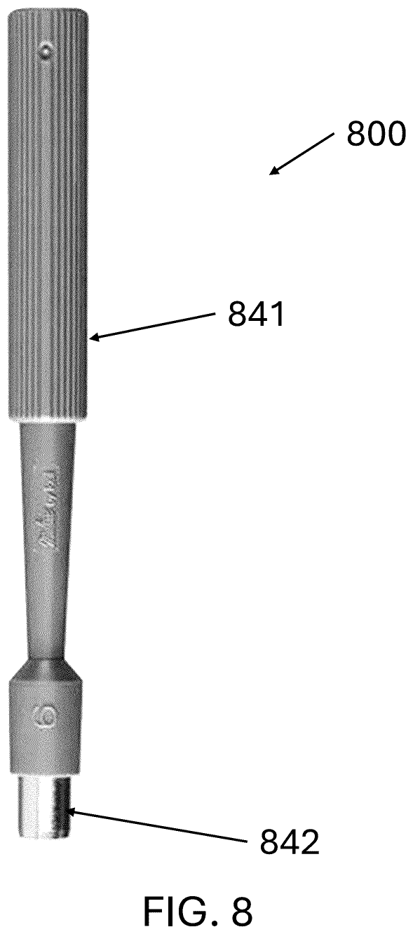

FIG. 8 shows an example trephine blade 800 that is used for surgical removal of certain tissues in a curved fashion such as corneal tissue of the eye. The trephine blade includes the handle 841 and the circular blade 842. Circular blade 842 may be used with the implant cutting region 700 to make the circular cut described above.

Wall Forceps that May be Used in Conjunction with the Pedestal

To ensure a repeatable, uniform, and clean detachment of the removable handle portion of the implant, the implant must be securely held while the cut is made. Regular forceps provides only a small surface area of contact and the use of a surgical blade or scalpel applies torque or twisting force that may result in uneven or undesired angle of the cut. Therefore a new tool to securely hold the implant during cutting is desired.

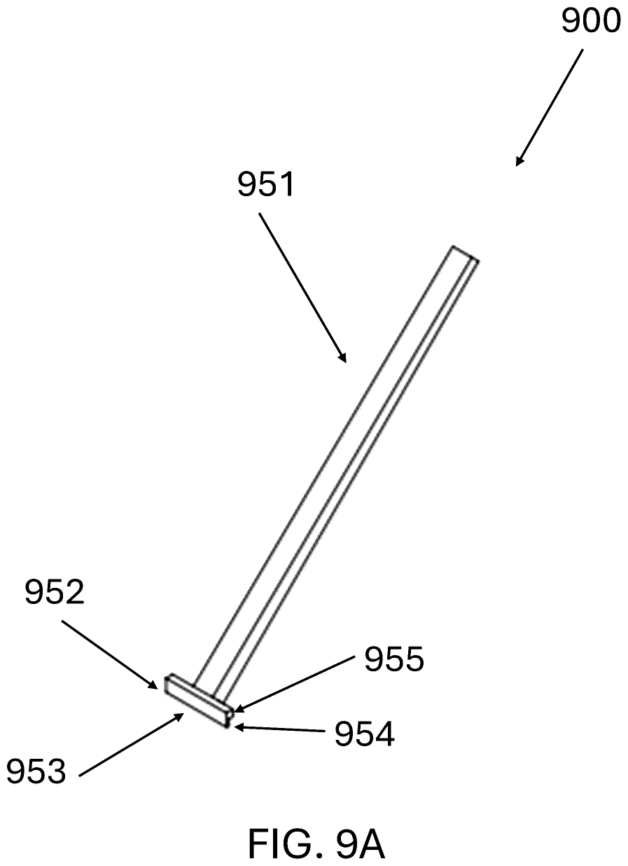

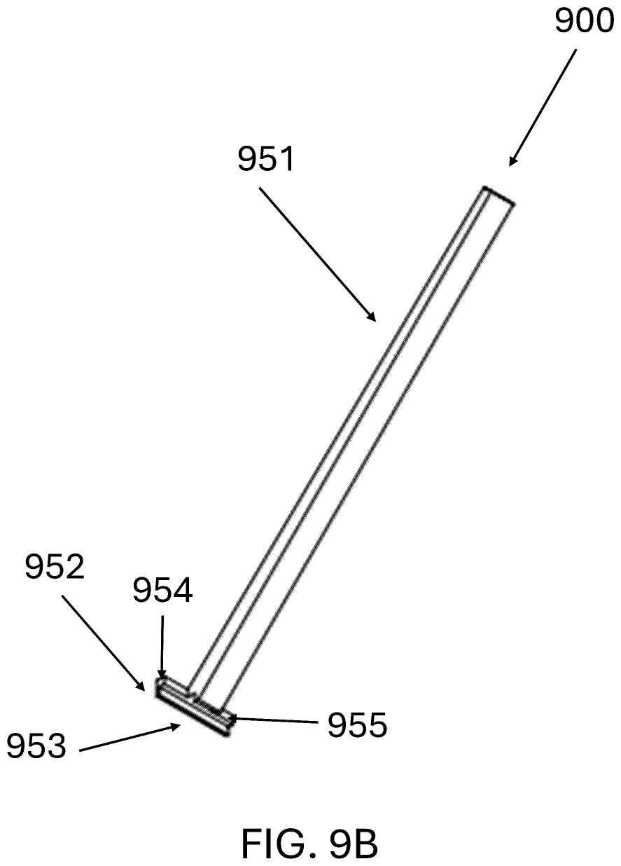

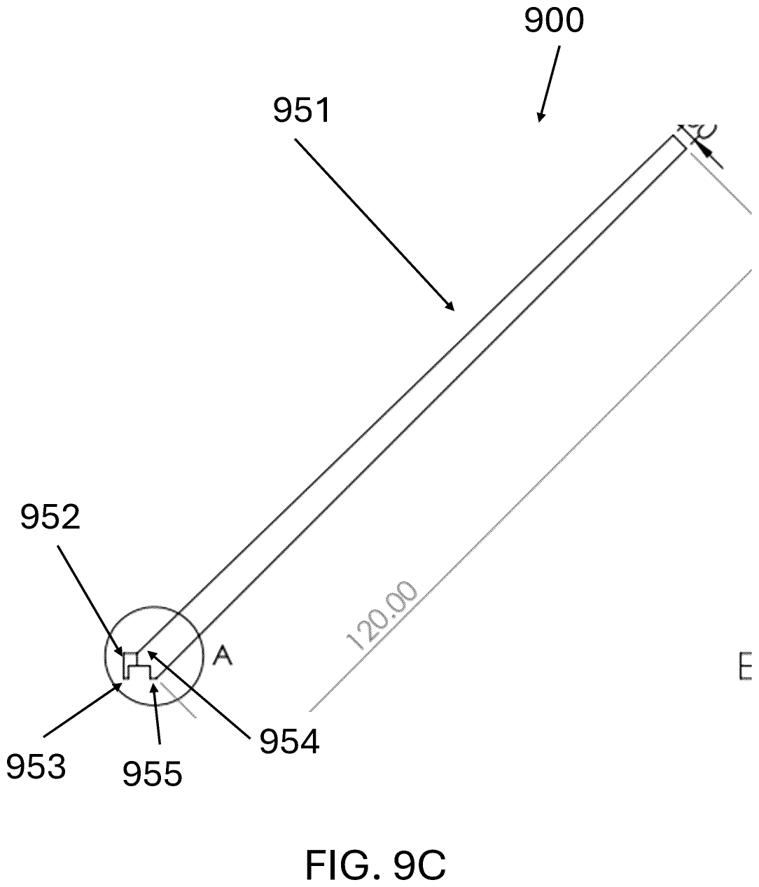

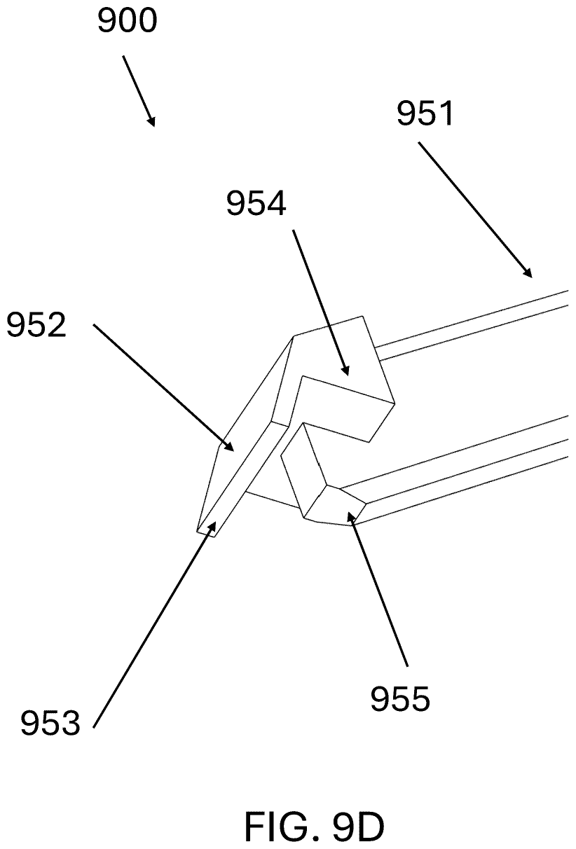

FIG. 9A-D shows a flat bottom wall forceps 900 including a flat surface 952 that, in some embodiments, is configured to serve the purposes of holding the implant against the pedestal and providing a wall to guide the cutting blade (e.g., during cutting of handle 140 from implant 100). The bottom surface 953 of the forceps is preferably coated with a thin layer of silicone or other coating to enhance stiction to further securely hold the implant. However, care must be taken to ensure the silicone coating is not cut off while detaching the implant handle. The silicone coating is ideally printed or painted on to minimize any silicone on the side walls 954 of the forceps. The handle 951 of the wall forceps 900 is ideally ergonomic and long enough to allow for visualization of the implant without having the handle or the user's hands blocking the line of sight.

FIG. 9C shows a flat bottom wall forceps with two parallel contacting flat bottom surfaces 953 in a toe/heel concept. By having two parallel flat bottom surfaces 953, the implant 100 is held even more securely and maintains contact with the pedestal 400 during the cutting process. In this embodiment, the handle 951 is at a 45 degree orientation compared to the pedestal 400 floor when the two flat surfaces are holding down the implant. Changing this particular angle does not compromise the functionality of the device, thus it can be modified for any application suitable for the wall forceps 900. The distance between the two contacting surfaces of toe and heel may be of a specific distance to align with a specific structure of the implant to replicate the cut in the same position. For example, the heel flat surface may be aligned and parallel to the identification plate embedded in the handle, thereby acting as a distance marker to the toe point where the cut is to be made.

FIG. 9D depicts an alternative toe/heel configuration embodiment. The bottom surface of the forceps 900 includes two contacting flat surfaces in a toe/heel concept. The heel surface 955 is a larger surface area that may be a trapezoidal shape, but alternatively a semi-sphere, square, or a variety of alternative shapes that provide a larger surface area instead of a thin strip, thereby providing a more secure hold by the heel surface 955.

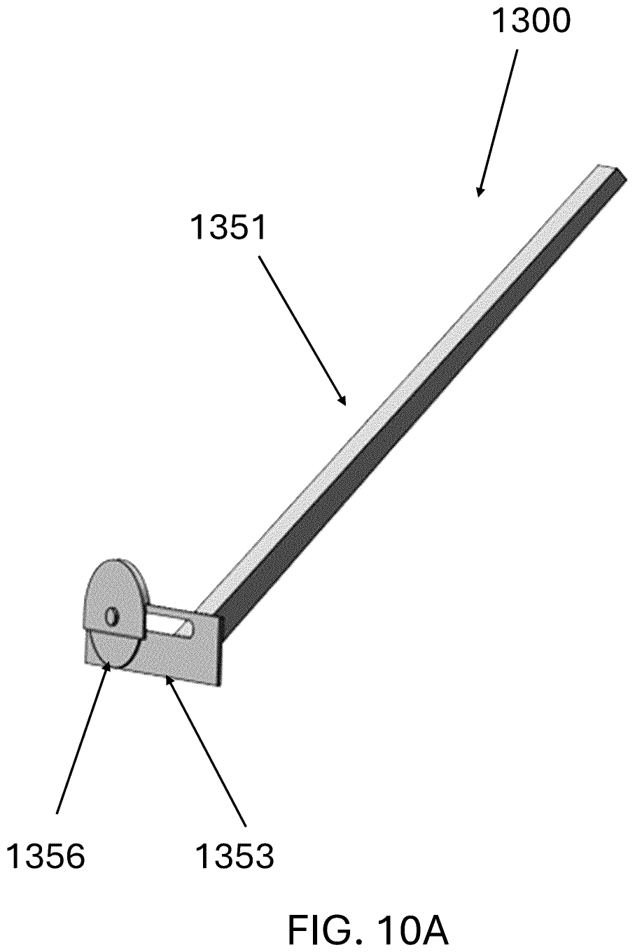

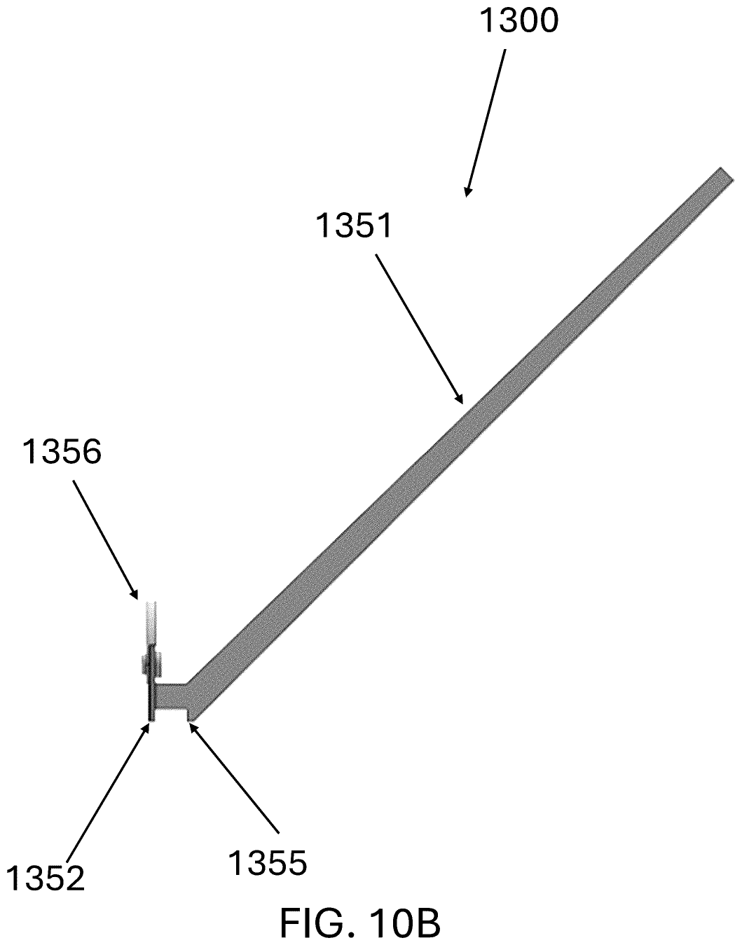

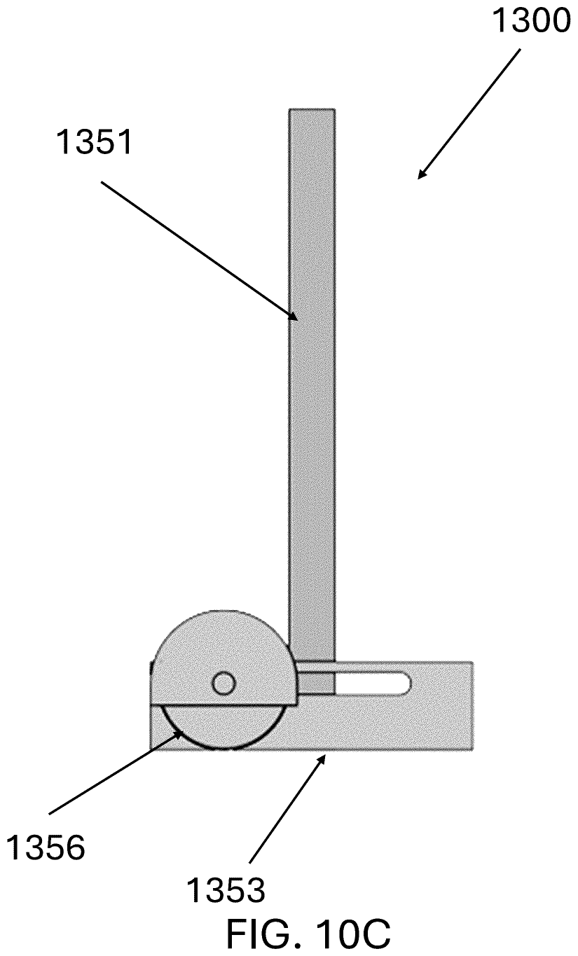

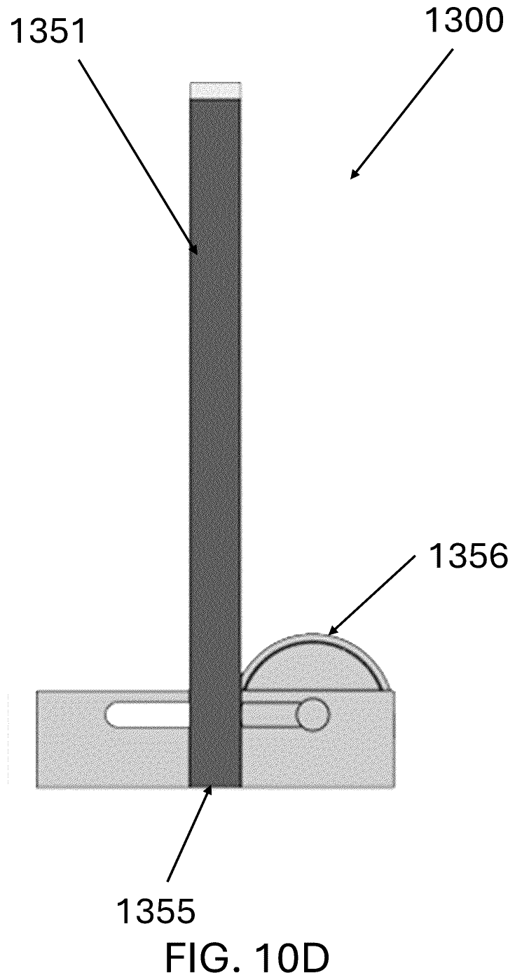

FIGS. 10A-10D depicts an embodiment in which a sliding cutting blade 1356 is integrated in the design of the wall forceps 1300. The sliding cutting blade 1356 may be used to cut handle 140 from implant 100 during use, for example. The sliding blade 1356, in some configurations, functions similar to a pizza-cutting disk blade in that a circular blade rotates on a central axis. Once the heel 1355 and toe 1353 are aligned in the desired position for handle removal, the circular blade 1356 is manually moved across the wall of the forceps to achieve the desired cut. There is a straight cutout for where the axis of the rotating blade 1356 would travel along the wall to facilitate this sliding motion. The rolling blade also has a cover facing away from the cutting end so that it can be safely handled by the user. This cut is done with a rolling blade as opposed to a straight 10-blade; however, this embodiment could be altered to function with a straight blade that does not rotate about a central axis. The blade can be circular or any shape—the main innovation of this embodiment is the sliding functionality of a blade mounted onto the wall forceps in an all-in-one fashion that enables positioning and cutting of the membrane using the same tool.

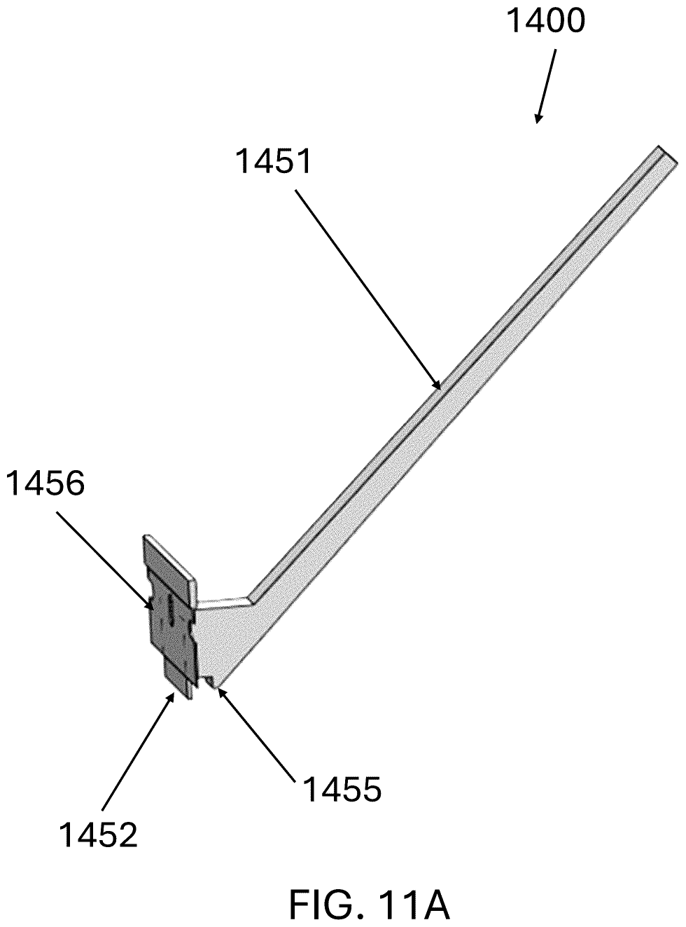

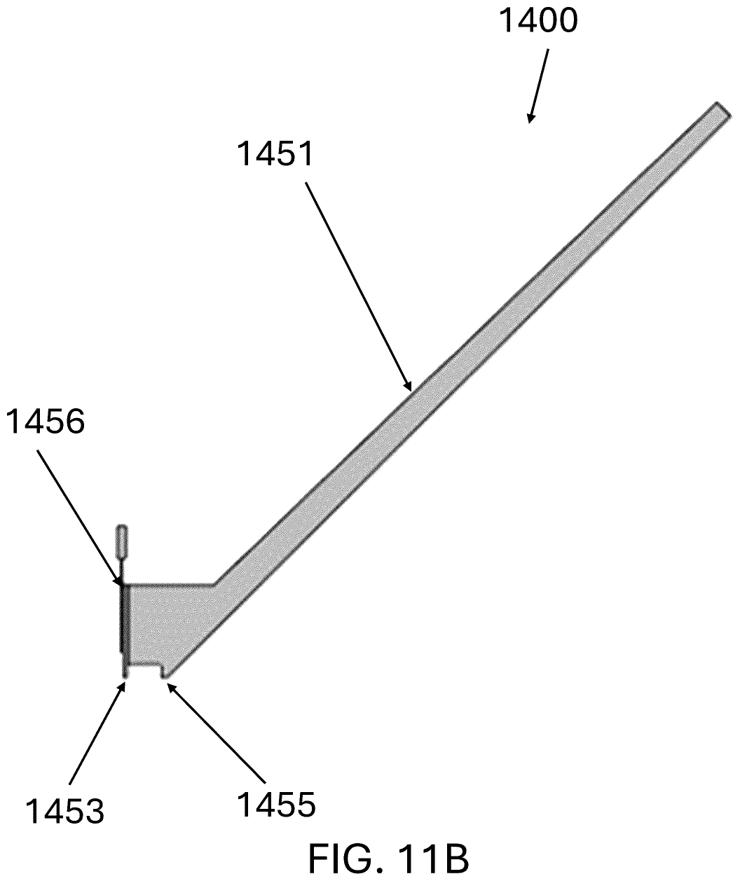

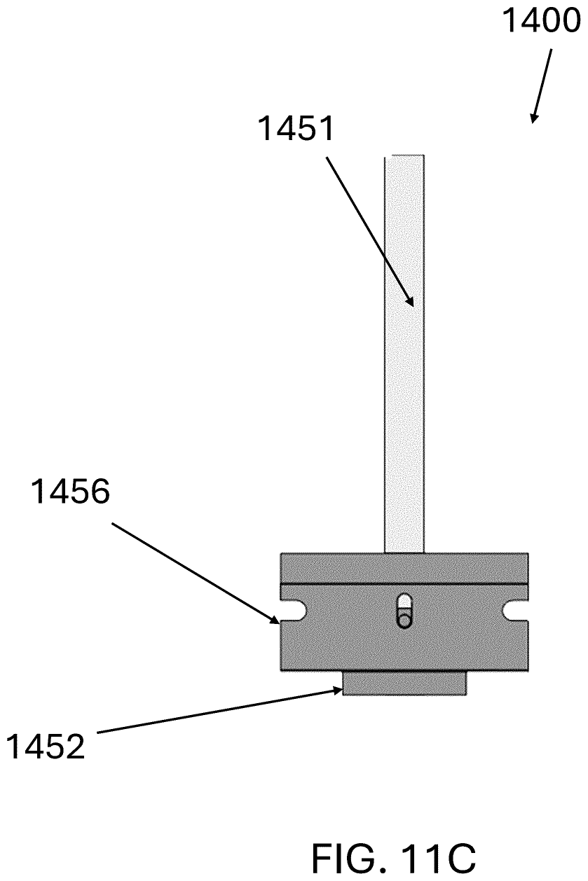

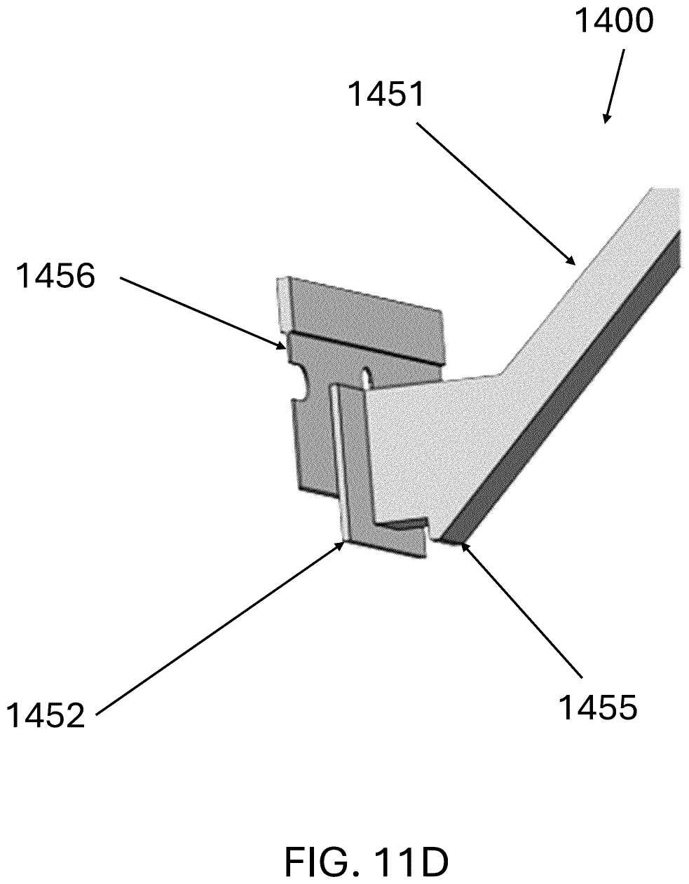

FIGS. 11A-11D depicts an embodiment in which the cutting blade 1456 is integrated in the design of the wall forceps 1400. In its standby position, a razorblade 1456 would be hovering over the pedestal cutting surface awaiting implant alignment. After alignment of the heel 1455 and toe 1453 on top of the handle of the implant 100, the razorblade 1456 would be pressed down on the implant 100 in the precise position between the head and tab to remove the handle. This rendition would cut the membrane by pressing down a blade 1456 onto the membrane instead of sliding it across the membrane, thereby reducing the probability of unwanted tearing caused by any dragging motion. After cutting is complete the blade could be retracted once more to yield the implant; handle, removed. An important advantage of this embodiment is the integration of positioning on the implant 100, holding down the implant, and cutting the implant all using a singular tool.

One or more of the above-mentioned tools may be combined into an implant processing kit to be used in a surgical suite to prepare a cell-seeded membrane for implantation. A kit according to certain embodiments may include any one or more of the tools described herein, e.g., wall forceps 900, 1300 and/or 1400. A kit may include one or more of the pedestals 400, 500, and/or 600 described herein, alone or in combination with the one or more tools. A single use kit of sterile tools and supplies designed to provide surgical staff with the materials required to safely and reproducibly remove the handle of an implant (e.g., a CPCB-RPE1 implant, an investigational product, and facilitate its handling prior to surgical delivery to the subretinal space). Each component of the kit can be specially chosen or custom designed to perform a specific function in handling the implant, removing its handle, and preparing it for implantation by, for example, a retinal surgeon. The kit, in some embodiments, can be supplied to clinical implantation sites separately from the cryopreserved cell-seeded membrane implant 100 and its subretinal delivery tool and stored at ambient temperature.

It will be appreciated by those skilled in the art that changes could be made to the exemplary embodiments shown and described above without departing from the broad inventive concepts thereof. It is understood, therefore, that this invention is not limited to the exemplary embodiments shown and described, but it is intended to cover modifications within the spirit and scope of the present invention as defined by the claims. For example, specific features of the exemplary embodiments may or may not be part of the claimed invention and various features of the disclosed embodiments may be combined. The words “right,” “left,” “lower,” and “upper,” designate directions in the drawings to which reference is made. Unless specifically set forth herein, the terms “a”, “an” and “the” are not limited to one element but instead should be read as meaning “at least one.”

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Further, to the extent that the methods of the present invention do not rely on the particular order of steps set forth herein, the particular order of the steps should not be construed as limitation on the claims. Any claims directed to the methods of the present invention should not be limited to the performance of their steps in the order written, and one skilled in the art can readily appreciate that the steps may be varied and still remain within the spirit and scope of the present invention.

What is claimed is:

1. A pedestal configured to receive an implantable substrate, the pedestal comprising:
a floor having a cutting region; and
a ramp nook having one or more sidewalls protruding from the floor, the one or more sidewalls at least partially defining a cavity that is sized and shaped to receive a first portion of the implantable substrate.

2. The pedestal of claim 1, wherein the cavity includes a sloped bottom surface that is angled obliquely relative to a plane of the floor.

3. The pedestal of claim 2, wherein the sloped bottom surface slopes below the plane of the floor to define a well in the floor.

4. The pedestal of claim 1, further comprising a dividing wall protruding from the floor and at least partially dividing the floor into the cutting region on a first side of the dividing wall and a washing region on a second side of the dividing wall.

5. The pedestal of claim 4, wherein the sidewalls extend from the dividing wall on the first side of the dividing wall.

6. The pedestal of claim 5, wherein cavity includes a sloped bottom surface that slopes upward as the slope bottom surface extends away from the dividing wall.

7. The pedestal of claim 1, wherein the cavity is sized and shaped to receive only a first portion of the implantable substrate while a second portion of the implantable substrate extends outside of the cavity into the cutting region.

8. The pedestal of claim 1, further comprising a perimeter containment wall surrounding the floor.

9. The pedestal of claim 8, wherein the pedestal is configured to contain a liquid in an area bounded by the perimeter containment wall.

10. The pedestal of claim 9, wherein the perimeter containment wall is of a height sufficient to contain a volume of liquid to fully submerge the implantable substrate.

11. The pedestal of claim 10, wherein the perimeter containment wall includes indicia for identifying the volume of liquid contained in the pedestal.

12. The pedestal of claim 1, wherein the floor and ramp nook are integrally formed from a common material.

13. The pedestal of claim 1, further comprising one or more ports in the floor, the one or more ports configured to introduce or remove liquid from pedestal.

14. The pedestal of claim 13, wherein the one or more ports can be opened and closed.

15. A kit comprising the pedestal of claim 1, and one or more instruments for handling the implantable membrane.

16. The kit of claim 15, wherein the one or more instruments includes forceps.

17. The kit of claim 15, wherein the one or more instruments includes a cutting tool.

18. The kit of claim 15, wherein the one or more instruments includes forceps having a cutting blade.

19. A method for preparing an implantable substrate for implantation comprising:
providing a pedestal including:
a floor having a cutting region; and
a ramp nook having one or more sidewalls protruding from the floor, the one or more sidewalls at least partially defining a cavity;
placing a first portion of the implantable substrate within the cavity while a second portion of the implantable substrate extends outside of the cavity into the cutting region; and
cutting the second portion of the implantable substrate from the first portion of the implantable substrate while the first portion of the implantable substrate is within the cavity.

20. The method of claim 19, further comprising, prior to cutting the second portion of the implantable substrate:
thawing the implantable substrate;
submerging the implantable substrate in a liquid contained on the floor of the pedestal; and
rising cryoprotectant from the implantable substrate with the liquid.
